(19) United States
(12) Patent Application Publication
(10) Pub. No.: US 2005/0053988 A1
(43) Pub. Date: Mar. 10, 2005

(54) Gene expressed in breast cancer and methods of use

(75) Inventors: Ira H. Pastan, Potomac, MD (US); Kristi Egland, Sioux Falls, SD (US); Byungkook Lee, Potomac, MD (US); James Vincent, Takoma Park, MD (US)

Correspondence Address: KLARQUIST SPARKMAN, LLP, 121 S.W. SALMON STREET, SUITE #1600, ONE WORLD TRADE CENTER, PORTLAND, OR, 97204-2988, US

(73) Assignee: The Gov. of the USA as represented by the Secretary of the Dept. of Health & Human Services

(21) Appl. No.: 10/913,196

(22) Filed: Aug. 5, 2004

Related U.S. Application Data
(60) Provisional application No. 60/493,522, filed on Aug. 8, 2003.

(51) Int. Cl. C12Q 1/00; C12Q 1/68; G01N 33/574; C07K 14/47

(52) U.S. Cl. 435/6; 435/7.23; 435/69.1; 435/320.1; 435/325; 530/350; 530/388.8; 536/23.5

(57) ABSTRACT

A polypeptide is disclosed that is detected in the breast cancer cells termed 68h05. Polynucleotides encoding 68h05 are also disclosed, as are vectors including these polynucleotides. Host cells transformed with these polynucleotides are also disclosed. Antibodies and immunoconjugates are disclosed that specifically bind 68h05. Methods are disclosed for using a 68h05 polypeptide, an antibody that specifically binds 68h05, or a polynucleotide encoding 68h05, such as in the treatment of breast cancer or prostate cancer. Assays are disclosed for the detection breast or prostate cancer. Pharmaceutical compositions including a 68h05 polypeptide, an antibody that specifically binds 68h05, or a polynucleotide encoding 68h05 are also disclosed. These pharmaceutical compositions are of use in the treatment of breast or prostate cancer.

### CROSS REFERENCE TO RELATED APPLICATIONS

This claims the benefit of U.S. Provisional Application No. 60/493,522, filed Aug. 8, 2003, which is incorporated by reference herein in its entirety.

### FIELD OF THE INVENTION

This disclosure relates to breast cancer and prostate cancer, specifically to polypeptides expressed in breast and prostate cancer cells that can be used in the detection and treatment of breast and/or prostate cancer.

### BACKGROUND OF THE INVENTION

Breast cancer is the most common type of epithelial cancer among women in the United States. More than 180,000 women are diagnosed with breast cancer each year. About 1 in 8 women in the United States (approximately 12.8 percent) will develop breast cancer during her lifetime. At present there are no curative therapies available for breast cancer that has metastasized from its site of origination. In addition, there is a need for diagnostic markers of use in the detection and staging breast cancers.

Cancer of the prostate is the most commonly diagnosed cancer in men and is the second most common cause of cancer death (Carter and Coffey, Prostate 16:39-48, 1990; Armbruster et al., Clinical Chemistry 39:181, 1993). If detected at an early stage, prostate cancer is potentially curable. However, a majority of cases are diagnosed at later stages when metastasis of the primary tumor has already occurred (Wang et al., Meth. Cancer Res. 19:179, 1982). Even early diagnosis is problematic because not all individuals who test positive in these screens develop cancer.

Present treatment for prostate cancer includes radical prostatectomy, radiation therapy, or hormonal therapy. With surgical intervention, complete eradication of the tumor is not always achieved and the observed re-occurrence of the cancer (12-68%) is dependent upon the initial clinical tumor stage (Zietman et al., Cancer 71:959, 1993). Thus, alternative methods of treatment including prophylaxis or prevention are desirable.

Immunotherapy is a potent new weapon against cancer. Immunotherapy involves evoking an immune response against cancer cells based on their production of target antigens. Immunotherapy based on cell-mediated immune responses involves generating a cell-mediated response to cells that produce particular antigenic determinants, while immunotherapy based on humoral immune responses involves generating specific antibodies to cells that produce particular antigenic determinants.

Cancer cells produce various proteins that can become the target of immunotherapy; antigenic determinants normally present on a specific cell type can also be immunogenic. For example, it has been shown that tumor infiltrating lymphocytes target and recognize antigenic determinants of the protein MART-1, produced by both normal melanocytes and malignant melanoma cells. Furthermore, active or passive immunotherapy directed against MART-1 or peptides of it that bind to MHC Class I molecules (epitopes of HLA A2, in particular) results in the destruction of melanoma cells as well as normal cells that produce MART-1 (Kawakami et al., J. Immunol. 21:237, 1998). As disclosed herein, discovery of breast cancer antigens and prostate cancer antigens can similarly be used to design immunotherapy methods for breast and/or prostate cancer.

### SUMMARY OF THE INVENTION

A new polypeptide is disclosed herein that is specifically detected in breast cancer cells. This polypeptide is termed 68h05 (but has also been called BPSR). Polynucleotides encoding 68h05 are also disclosed herein, as are vectors including polynucleotides encoding 68h05, and host cells transformed with these polynucleotides. Antibodies that specifically bind 68h05 are also disclosed.

Methods for using a 68h05 polypeptide, an antibody that specifically binds 68h05, or a polynucleotide encoding 68h05 are also disclosed. Methods of use include an assay to detect breast or prostate cancer, or the use of these compositions to treat breast cancer or prostate cancer. Also disclosed are pharmaceutical compositions including a 68h05 polypeptide, an antibody that specifically binds 68h05, or a polynucleotide encoding 68h05.

The foregoing and other features and advantages will become more apparent from the following detailed description of several embodiments, which proceeds with reference to the accompanying figures.

### BRIEF DESCRIPTION OF THE FIGURES

FIG. 1 is a schematic diagram showing the alignment of the 68h05 consensus sequence and MAPcL clone cDNA sequences with the human genome chromosome 9 (UCSChg15). Black boxes represent individual MAPcL clones. Primer locations are indicated by arrows labeled F (forward) 68_h05-C-For (5′-CTCTAGACGCGCTGCACCTGC -3′), SEQ ID NO: 8, and R (reverse) 68_h05-C-Rev (5′-CCGCTGCCGCCCAAGCCTG-3′, SEQ ID NO: 9).

FIGS. 2A-2B shows the nucleotide and amino acid sequence of 68h05. FIG. 2A is the nucleic acid sequence of 68h05. The sequence is 1439 base pairs in length. The open reading frame with stop codon is shown in bold. FIG. 2B is the amino acid sequence of 68h05. The translated open reading frame is 383 amino acids.

FIG. 3 is a schematic diagram showing the predicted structure of 68h05. The 68h05 protein contains a signal peptide, leucine-rich-repeat (LRR) domains with corresponding N- and C-terminal LRR domains, and a transmembrane domain. The transmembrane domain spans amino acids 265-287. The protein is 383 amino acids in length. The N-terminal and C-terminal amino acid for each domain is indicated in the following Table:

| Name | begin | end |
|---|---|---|
| signal sequence | 1 | 26 |
| LRRNT | 42 | 75 |
| LRR_TYP | 72 | 93 |
| LRR_TYP | 95 | 117 |
| LRR_TYP | 118 | 141 |
| LRR_TYP | 142 | 165 |
| LRR_TYP | 166 | 189 |
| LRRCT | 201 | 254 |
| transmembrane | 265 | 287 |

The signal sequence was predicted by SignalP program. All other protein domains were predicted by SMART.

FIGS. 4A-4B are digital images showing the expression of 68h05 in the MAPcL cell lines and breast cancers. FIG. 4A shows the expression of 68h05 in the MAPcL cell lines. Expression levels were determined by RT-PCR using total RNA isolated from the library cell lines as a template for cDNA synthesis. PCR was performed by using primers to 68h05 (shown in FIG. 1). The PCR products were analyzed on a 1.5% agarose gel with ethidium bromide staining as follows: lane 1, LNCaP; lane 2, MCF7; lane 3, SK-BR-3; lane 4, ZR-75-1; lane 5, MDA-MB-231; lane 6, hTERT-HME1; lane 7, pKAE68h05 and lane 8, no template. The quality of the generated cDNA was verified with separate PCRs using actin primers which amplify a 640-bp fragment. The DNA ladder in base pairs is indicated on the right. FIG. 4B is a digital image of a RT-PCR analysis showing expression of 68h05 in breast cancer samples. RT-PCR analysis was performed by using 21 breast cancer total RNA samples as templates for cDNA synthesis. The 68h05 PCR primers, shown in FIG. 1, amplify a 430-bp fragment. The PCR products were analyzed on a 1.5% agarose gel with ethidium bromide staining as follows: lanes 1-21, breast tumors; lane 22, pKAE68h05 as a positive control; lane 23, empty; lane 24, water as a negative control. PCRs using actin primers were performed separately and produced a 640-bp product. The DNA ladder in base pairs is indicated on the right.

FIG. 5 is a digital image showing the expression of 68h05 in 24 normal tissues. RT-PCRs were performed by using a rapid-scan gene expression panel containing cDNA samples from 24 different normal tissues: 1, brain; 2, heart; 3, kidney; 4, spleen; 5, liver; 6, colon; 7, lung; 8,.small intestine; 9, muscle; 10, stomach; 11, testis; 12, placenta; 13, salivary gland; 14, thyroid; 15, adrenal gland; 16, pancreas; 17, ovary; 18, uterus; 19, prostate; 20, skin; 21, peripheral blood lymphocyte; 22, bone marrow; 23, fetal brain; 24, fetal liver; and 25, pKAE68h05. Note the faint band in lane 19, prostate. PCR primers for 68h05 are shown in FIG. 1. These primers are located in different exons and amplify a 430-bp 68h05 fragment. As a positive control, pKAE68h05 was used as a template for the PCR (lane 25). PCRs using actin primers were performed separately and produced a 640-bp product. The PCR products were analyzed on a 1.5% agarose gel with ethidium bromide staining.

FIG. 6 is a digital image of an in vitro Translation of Clone 68h05. The cDNA clone 68h05 in plasmid vector pCMVSport6.0 was transcribed and translated in vitro using the TNT® Coupled Reticulocyte Lysate System. Lane 1: 68h06 product, expected molecular weight about 35 kDa, Lane 2: 19 kDa protein, Lane 3: negative control, Lane 4: empty, Lane 5: 68h06 product, expected molecular weight about 35 kDa. The bands observed at 12000 Da are globin. The bands observed at ˜25 kDa are aminoacyl tRNAs.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. In the accompanying sequence listing:

SEQ ID NO: 1 is an amino acid sequence of a 68h05 polypeptide.

SEQ ID NO: 2 is a nucleic acid sequence of a polynucleotide encoding 68h05.

SEQ ID NOs: 3-7 are the amino acid sequences of fragments of 68h05 that are predicted to specifically bind MHC Class I.

SEQ ID NO: 8 is the nucleic acid sequence of the F primer.

SEQ ID NO: 9 is the nucleic acid sequence of the R primer.

SEQ ID NOs: 10-11 are the nucleic acid sequences of primers used in the experiments described herein.

SEQ ID NO: 12 is the amino acid sequence of an immunogenic 68h05 peptide.

### DETAILED DESCRIPTION

A novel gene product expressed in breast cancer cells, termed 68h05 (or BPSR), is disclosed herein.

After defining some of the terms used herein, the discussion below sets forth the 68h05 protein, nucleic acid sequences encoding 68h05, and the expression of this protein. As 68h05 is expressed in breast cancer, it is of use in detecting breast cancer cells. As 68h05 is also expressed in a prostate cancer cell line, it is of use in detecting prostate cancer. Diagnostic kits for 68h05 are thus disclosed.

Antibodies that specifically bind 68h05 are also disclosed herein. These antibodies are of use in detection assays, as well as in the production of immunoconjugates, such as immunotoxins, which can be used to target breast or prostate cancer.

Nucleic acids encoding 68h05, or a 68h05 polypeptide, can be used to produce an immune response against breast cancer cells. Thus, pharmaceutical compositions including 68h05, or a nucleic acid encoding 68h05, are also disclosed.

Methods for treating breast and prostate cancer using the compositions disclosed herein are also disclosed.

I. Abbreviations

CTL: cytotoxic T lymphocyte

DNA: deoxyribonucleic acid

DT: diphtheria toxin

EM: effector molecule

LRR: leucine rich region.

MHC: Major Histocompatibility Complex

MAPcL: Membrane Associated Polyribosomal cDNA Library

RT-PCR: reverse transcriptase polymerase chain reaction

RNA: ribonucleic acid

PCR: polymerase chain reaction

PE: Pseudomonas exotoxin

II. Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of this disclosure, the following explanations of specific terms are provided:

Antigen: A compound, composition, or substance that can stimulate the production of antibodies or a T cell response in an animal, including compositions that are injected or absorbed into an animal. An antigen reacts with the products of specific humoral or cellular immunity, including those induced by heterologous immunogens. The term “antigen” includes all related antigenic epitopes. “Epitope” or “antigenic determinant” refers to a site on an antigen to which B and/or T cells respond. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least three, and more usually, at least five or eight to ten amino acids in a unique spatial conformation. Methods of determining spatial conformation of epitopes include, for example, x-ray crystallography and 2-dimensional nuclear magnetic resonance.

An antigen can be a tissue-specific antigen, or a disease-specific antigen. These terms are not exclusive, as a tissue-specific antigen can also be a disease specific antigen. A tissue-specific antigen is expressed in a limited number of tissues, such as a single tissue. Specific, non-limiting examples of a tissue specific antigen are a breast specific antigen, or a prostate specific antigen. A disease-specific antigen is expressed coincidentally with a disease process. Specific non-limiting examples of a disease-specific antigen are an antigen whose expression correlates with, or is predictive of, tumor formation, such as breast cancer or prostate cancer. A disease specific antigen may be an antigen recognized by T cells or B cells.

Amplification: Of a nucleic acid molecule (e.g., a DNA or RNA molecule) refers to use of a technique that increases the number of copies of a nucleic acid molecule in a specimen. An example of amplification is the polymerase chain reaction, in which a biological sample collected from a subject is contacted with a pair of oligonucleotide primers, under conditions that allow for the hybridization of the primers to a nucleic acid template in the sample. The primers are extended under suitable conditions, dissociated from the template, and then re-annealed, extended, and dissociated to amplify the number of copies of the nucleic acid. The product of amplification may be characterized by electrophoresis, restriction endonuclease cleavage patterns, oligonucleotide hybridization or ligation, and/or nucleic acid sequencing using standard techniques. Other examples of amplification include strand displacement amplification, as disclosed in U.S. Pat. No. 5,744,311; transcription-free isothermal amplification, as disclosed in U.S. Pat. No. 6,033,881; repair chain reaction amplification, as disclosed in WO 90/01069; ligase chain reaction amplification, as disclosed in EP-A-320 308; gap filling ligase chain reaction amplification, as disclosed in U.S. Pat. No. 5,427,930; and NASBA™ RNA transcription-free amplification, as disclosed in U.S. Pat. No. 6,025,134.

Animal: Living multi-cellular vertebrate organisms, a category that includes, for example, mammals and birds. The term mammal includes both human and non-human mammals. Similarly, the term “subject” includes both human and veterinary subjects.

Breast cancer: A neoplastic condition of breast tissue that can be benign or malignant. The most common type of breast cancer is ductal carcinoma. Ductal carcinoma in situ is a non-invasive neoplastic condition of the ducts. Lobular carcinoma is not an invasive disease but is an indicator that a carcinoma may develop. Infiltrating (malignant) carcinoma of the breast can be divided into stages (I, IIA, IIB, IIIA, IIIB, and IV).

Chemotherapeutic agents: Any chemical agent with therapeutic usefulness in the treatment of diseases characterized by abnormal cell growth. Such diseases include tumors, neoplasms, and cancer as well as diseases characterized by hyperplastic growth such as psoriasis. In one embodiment, a chemotherapeutic agent is an agent of use in treating breast and/or prostate cancer. In one embodiment, a chemotherapeutic agent is radioactive compound. One of skill in the art can readily identify a chemotherapeutic agent of use (e.g. see Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2^{nd} ed., © 2000 Churchill Livingstone, Inc; Baltzer L, Berkery R (eds): Oncology Pocket Guide to Chemotherapy, 2nd ed. St. Louis, Mosby-Year Book, 1995; Fischer D S, Knobf M F, Durivage H J (eds): The Cancer Chemotherapy Handbook, 4th ed. St. Louis, Mosby-Year Book, 1993). Combination chemotherapy is the administration of more than one agent to treat cancer, such as the administration of antibodies to 68h05 in combination with a radioactive or chemical compound to a subject.

Conservative variants: “Conservative” amino acid substitutions are those substitutions that do not substantially affect or decrease an activity or antigenicity of 68h05. Specific, non-limiting examples of a conservative substitution include the following examples:

| Original Residue | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

The term conservative variation also includes the use of a substituted amino acid in place of an unsubstituted parent amino acid, provided that antibodies raised to the substituted polypeptide also immunoreact with the unsubstituted polypeptide. Non-conservative substitutions are those that reduce an activity or antigenicity.

cDNA (complementary DNA): A piece of DNA lacking internal, non-coding segments (introns) and regulatory sequences that determine transcription. cDNA is synthesized in the laboratory by reverse transcription from messenger RNA extracted from cells.

Degenerate variant: A polynucleotide encoding a 68h05 polypeptide that includes a sequence that is degenerate as a result of the genetic code. There are 20 natural amino acids, most of which are specified by more than one codon. Therefore, all degenerate nucleotide sequences are included in this disclosure as long as the amino acid sequence of the 68h05 polypeptide encoded by the nucleotide sequence is unchanged.

Diagnostic: Identifying the presence or nature of a pathologic condition, such as, but not limited to, breast cancer or prostate cancer. Diagnostic methods differ in their sensitivity and specificity. The “sensitivity” of a diagnostic assay is the percentage of diseased individuals who test positive (percent of true positives). The “specificity” of a diagnostic assay is one minus the false positive rate, where the false positive rate is defined as the proportion of those without the disease who test positive. While a particular diagnostic method may not provide a definitive diagnosis of a condition, it suffices if the method provides a positive indication that aids in diagnosis. “Prognostic” is the probability of development (e.g., severity) of a pathologic condition, such as breast cancer or prostate cancer, or metastasis.

Epitope: An antigenic determinant. These are particular chemical groups or peptide sequences on a molecule that are antigenic, i.e. that elicit a specific immune response. An antibody specifically binds a particular antigenic epitope on a polypeptide. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least five or eight to ten amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus expression control sequences can include appropriate promoters, enhancers, transcription terminators, a start codon (i.e., ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see e.g., Bitter et al., Methods in Enzymology 153:516-544, 1987). For example, when cloning in bacterial systems, inducible promoters such as pL of bacteriophage lambda, plac, ptrp, ptac (ptrp-lac hybrid promoter) and the like may be used. In one embodiment, when cloning in mammalian cell systems, promoters derived from the genome of mammalian cells (e.g., metallothionein promoter) or from mammalian viruses (e.g., the retrovirus long terminal repeat; the adenovirus late promoter; the vaccinia virus 7.5K promoter) can be used. Promoters produced by recombinant DNA or synthetic techniques may also be used to provide for transcription of the nucleic acid sequences.

Host cells: Cells in which a vector can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

Immune response: A response of a cell of the immune system, such as a B cell, T cell, or monocyte, to a stimulus. In one embodiment, the response is specific for a particular antigen (an “antigen-specific response”). In one embodiment, an immune response is a T cell response, such as a CD4+ response or a CD8+ response. In another embodiment, the response is a B cell response, and results in the production of specific antibodies.

Immunoconjugate: A covalent linkage of an effector molecule to an antibody. The effector molecule can be a toxin. Specific, non-limiting examples of toxins include, but are not limited to, abrin, ricin, Pseudomonas exotoxin (PE, such as PE35, PE37, PE38, and PE40), diphtheria toxin (DT), saporin, restrictocin, or modified toxins thereof, or other toxic agents that directly or indirectly inhibit cell growth or kill cells. For example, PE and DT are highly toxic compounds that typically bring about death through liver and heart toxicity in humans. PE and DT, however, can be modified into a form for use as an immunotoxin by removing the native targeting component of the toxin (e.g., domain Ia of PE and the B chain of DT) and replacing it with a different targeting moiety, such as an antibody. A “chimeric molecule” is a targeting moiety, such as a ligand or an antibody, conjugated (coupled) to an effector molecule. The term “conjugated” or “linked” refers to making two polypeptides into one contiguous polypeptide molecule. In one embodiment, an antibody is joined to an effector molecule (EM). In another embodiment, an antibody joined to an effector molecule is further joined to a lipid or other molecule to a protein or peptide to increase its half-life in the body. The linkage can be either by chemical or recombinant means. In one embodiment, the linkage is chemical, wherein a reaction between the antibody moiety and the effector molecule has produced a covalent bond formed between the two molecules to form one molecule. A peptide linker (short peptide sequence) can optionally be included between the antibody and the effector molecule.

Immunogenic peptide: A peptide which comprises an allele-specific motif or other sequence such that the peptide will bind an MHC molecule and induce a cytotoxic T lymphocyte (“CTL”) response, or a B cell response (e.g. antibody production) against the antigen from which the immunogenic peptide is derived.

In one embodiment, immunogenic peptides are identified using sequence motifs or other methods, such as neural net or polynomial determinations, known in the art. Typically, algorithms are used to determine the “binding threshold” of peptides to select those with scores that give them a high probability of binding at a certain affinity and will be immunogenic. The algorithms are based either on the effects on MHC binding of a particular amino acid at a particular position, the effects on antibody binding of a particular amino acid at a particular position, or the effects on binding of a particular substitution in a motif-containing peptide. Within the context of an immunogenic peptide, a “conserved residue” is one which appears in a significantly higher frequency than would be expected by random distribution at a particular position in a peptide. In one embodiment, a conserved residue is one where the MHC structure may provide a contact point with the immunogenic peptide. MHC binding prediction programs are available on the internet, such as ProPed-I, located on the imtech website (Singh and Raghava, ProPred1: Prediction of promiscuous MHC class-I binding sites, Bioinformatics, 2003).

Immunogenic composition: A composition comprising a 68h05 polypeptide that induces a measurable immune response against cells expressing 68h05 polypeptide, such as a measurable CTL response against cells expressing 68h05 polypeptide, or a measurable B cell response (such as production of antibodies that specifically bind 68h05) against a 68h05 polypeptide. It further refers to isolated nucleic acids encoding a 68h05 polypeptide that can be used to express the 68h05 polypeptide (and elicit an immune response against 68h05). For in vitro use, the immunogenic composition may consist of the isolated protein or peptide. For in vivo use, the immunogenic composition will typically comprise the protein or peptide in pharmaceutically acceptable carriers, and/or other agents. Any particular peptide, 68h05 polypeptide, or nucleic acid encoding the polypeptide, can be readily tested for its ability to induce a CTL or B cell response by art-recognized assays.

Immunoglobulin (antibody): A protein including one or more polypeptides substantially encoded by immunoglobulin genes. The recognized immunoglobulin genes include the kappa, lambda, alpha (IgA), gamma (IgG₁, IgG₂, IgG₃, IgG₄), delta (IgD), epsilon (IgE) and mu (IgM) constant region genes, as well as the myriad immunoglobulin variable region genes. Full-length immunoglobulin light chains are generally about 25 Kd or 214 amino acids in length. Full-length immunoglobulin heavy chains are generally about 50 Kd or 446 amino acid in length. Light chains are encoded by a variable region gene at the NH2-terminus (about 110 amino acids in length) and a kappa or lambda constant region gene at the COOH-terminus. Heavy chains are similarly encoded by a variable region gene (about 116 amino acids in length) and one of the other constant region genes.

The basic structural unit of an antibody is generally a tetramer that consists of two identical pairs of immunoglobulin chains, each pair having one light and one heavy chain. In each pair, the light and heavy chain variable regions bind to an antigen, and the constant regions mediate effector functions. Immunoglobulins also exist in a variety of other forms including, for example, Fv, Fab, and (Fab′)₂, as well as bifunctional hybrid antibodies and single chains (e.g., Lanzavecchia et al., Eur. J. Immunol. 17:105, 1987; Huston et al., Proc. Natl. Acad. Sci. U.S.A., 85:5879-5883, 1988; Bird et al., Science 242:423-426, 1988; Hood et al., Immunology, Benjamin, N.Y., 2nd ed., 1984; Hunkapiller and Hood, Nature 323:15-16, 1986).

An immunoglobulin light or heavy chain variable region includes a framework region interrupted by three hypervariable regions, also called complementarity determining regions (CDR's) (see, Sequences of Proteins of Immunological Interest, E. Kabat et al., U.S. Department of Health and Human Services, 1983). As noted above, the CDRs are primarily responsible for binding to an epitope of an antigen.

Chimeric antibodies are antibodies whose light and heavy chain genes have been constructed, typically by genetic engineering, from immunoglobulin variable and constant region genes belonging to different species. For example, the variable segments of the genes from a mouse monoclonal antibody can be joined to human constant segments, such as kappa and gamma 1 or gamma 3. In one example, a therapeutic chimeric antibody is thus a hybrid protein composed of the variable or antigen-binding domain from a mouse antibody and the constant or effector domain from a human antibody, although other mammalian species can be used, or the variable region can be produced by molecular techniques. Methods of making chimeric antibodies are well known in the art, e.g., see U.S. Pat. No. 5,807,715.

A “humanized” immunoglobulin is an immunoglobulin including a human framework region and one or more CDRs from a non-human (such as a mouse, rat, or synthetic) immunoglobulin. The non-human immunoglobulin providing the CDRs is termed a “donor” and the human immunoglobulin providing the framework is termed an “acceptor.” In one embodiment, all the CDRs are from the donor immunoglobulin in a humanized immunoglobulin. Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDRs. The acceptor framework of a humanized immunoglobulin or antibody may have a limited number of substitutions by amino acids taken from the donor framework. Humanized or other monoclonal antibodies can have additional conservative amino acid substitutions which have substantially no effect on antigen binding or other immunoglobulin functions. Exemplary conservative substitutions are those such as gly, ala; val, ile, leu; asp, glu; asn, gln; ser, thr; lys, arg; and phe, tyr. Humanized immunoglobulins can be constructed by means of genetic engineering (e.g., see U.S. Pat. No. 5,585,089).

A human antibody is an antibody wherein the light and heavy chain genes are of human origin. Human antibodies can be generated using methods known in the art. Human antibodies can be produced by immortalizing a human B cell secreting the antibody of interest. Immortalization can be accomplished, for example, by EBV infection or by fusing a human B cell with a myeloma or hybridoma cell to produce a trioma cell. Human antibodies can also be produced by phage display methods (see, e.g., Dower et al., PCT Publication No. WO91/17271; McCafferty et al., PCT Publication No. WO92/001047; and Winter, PCT Publication No. WO92/20791, which are herein incorporated by reference), or selected from a human combinatorial monoclonal antibody library (see the Morphosys website). Human antibodies can also be prepared by using transgenic animals carrying a human immunoglobulin gene (for example, see Lonberg et al., PCT Publication No. WO93/12227; and Kucherlapati, PCT Publication No. WO91/10741, which are herein incorporated by reference).

Isolated: An “isolated” biological component (such as a nucleic acid or protein or organelle) has been substantially separated or purified away from other biological components in the cell of the organism in which the component naturally occurs, i.e., other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

Label: A detectable compound or composition that is conjugated directly or indirectly to another molecule to facilitate detection of that molecule. Specific, non-limiting examples of labels include fluorescent tags, enzymatic linkages, and radioactive isotopes.

Lymphocytes: A type of white blood cell that is involved in the immune defenses of the body. There are two main types of lymphocytes: B cells and T cells.

Mammal: This term includes both human and non-human mammals. Similarly, the term “subject” includes both human and veterinary subjects.

Monoclonal antibody: An antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

Oligonucleotide: A linear polynucleotide sequence of up to about 100 nucleotide bases in length.

Open reading frame (ORF): A series of nucleotide triplets (codons) coding for amino acids without any internal termination codons. These sequences are usually translatable into a peptide.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Peptide: A chain of amino acids of between 3 and 30 amino acids in length, such as from 8 to ten amino acids in length. In one embodiment, a peptide is from about 10 to about 25 amino acids in length. In yet another embodiment, a peptide is from about 11 to about 20 amino acids in length. In yet another embodiment, a peptide is about 12 amino acids in length.

Peptide Modifications: 68h05 polypeptides include synthetic embodiments of peptides described herein. In addition, analogues (non-peptide organic molecules), derivatives (chemically functionalized peptide molecules obtained starting with the disclosed peptide sequences) and variants (homologs) of these proteins can be utilized in the methods described herein. Each polypeptide of this disclosure is comprised of a sequence of amino acids, which may be either L- and/or D-amino acids, naturally occurring and otherwise.

Peptides may be modified by a variety of chemical techniques to produce derivatives having essentially the same activity as the unmodified peptides, and optionally having other desirable properties. For example, carboxylic acid groups of the protein, whether carboxyl-terminal or side chain, may be provided in the form of a salt of a pharmaceutically-acceptable cation or esterified to form a C₁-C₁₆ ester, or converted to an amide of formula NR₁R₂ wherein R₁ and R₂ are each independently H or C₁-C₁₆ alkyl, or combined to form a heterocyclic ring, such as a 5- or 6-membered ring. Amino groups of the peptide, whether amino-terminal or side chain, may be in the form of a pharmaceutically-acceptable acid addition salt, such as the HCl, HBr, acetic, benzoic, toluene sulfonic, maleic, tartaric and other organic salts, or may be modified to C₁-C₁₆ alkyl or dialkyl amino or further converted to an amide.

Hydroxyl groups of the peptide side chains may be converted to C₁-C₁₆ alkoxy or to a C₁-C₁₆ ester using well-recognized techniques. Phenyl and phenolic rings of the peptide side chains may be substituted with one or more halogen atoms, such as fluorine, chlorine, bromine or iodine, or with C₁-C₁₆ alkyl, C₁-C₁₆ alkoxy, carboxylic acids and esters thereof, or amides of such carboxylic acids. Methylene groups of the peptide side chains can be extended to homologous C₂-C₄ alkylenes. Thiols can be protected with any one of a number of well-recognized protecting groups, such as acetamide groups. Those skilled in the art will also recognize methods for introducing cyclic structures into the peptides of this disclosure to select and provide conformational constraints to the structure that result in enhanced stability.

Peptidomimetic and organomimetic embodiments are envisioned, whereby the three-dimensional arrangement of the chemical constituents of such peptido- and organomimetics mimic the three-dimensional arrangement of the peptide backbone and component amino acid side chains, resulting in such peptido- and organomimetics of a 68h05 polypeptide having measurable or enhanced ability to generate an immune response. For computer modeling applications, a pharmacophore is an idealized three-dimensional definition of the structural requirements for biological activity. Peptido- and organomimetics can be designed to fit each pharmacophore with current computer modeling software (using computer assisted drug design or CADD). See Walters, “Computer-Assisted Modeling of Drugs,” in Klegerman & Groves, eds., 1993, Pharmaceutical Biotechnology, Interpharm Press: Buffalo Grove, Ill., pp. 165-174 and Principles of Pharmacology Munson (ed.) 1995, Ch. 102, for descriptions of techniques used in CADD. Also included are mimetics prepared using such techniques.

Pharmaceutically acceptable carriers: The pharmaceutically acceptable carriers of use are conventional. Remington 's Pharmaceutical Sciences, by E. W. Martin, Mack Publishing Co., Easton, Pa., 15th Edition (1975), describes compositions and formulations suitable for pharmaceutical delivery of the fusion proteins herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Polynucleotide: The term polynucleotide or nucleic acid sequence refers to a polymeric form of nucleotide at least ten bases in length. A recombinant polynucleotide includes a polynucleotide that is not immediately contiguous with both of the coding sequences with which it is immediately contiguous (one on the 5′ end and one on the 3′ end) in the naturally occurring genome of the organism from which it is derived. The term therefore includes, for example, a recombinant DNA which is incorporated into a vector; into an autonomously replicating plasmid or virus; or into the genomic DNA of a prokaryote or eukaryote, or which exists as a separate molecule (e.g., a cDNA) independent of other sequences. The nucleotides can be ribonucleotides, deoxyribonucleotides, or modified forms of either nucleotide. The term includes single- and double-stranded forms of DNA.

Polypeptide: Any chain of amino acids, regardless of length or post-translational modification (e.g., glycosylation or phosphorylation). In one embodiment, the polypeptide is 68h05 polypeptide.

Probes and primers: A probe comprises an isolated nucleic acid attached to a detectable label or reporter molecule. Primers are short nucleic acids, preferably DNA oligonucleotides, 15 nucleotides or more in length. Primers may be annealed to a complementary target DNA strand by nucleic acid hybridization to form a hybrid between the primer and the target DNA strand, and then extended along the target DNA strand by a DNA polymerase enzyme. Primer pairs can be used for amplification of a nucleic acid sequence, e.g., by the polymerase chain reaction (PCR) or other nucleic acid amplification methods known in the art. One of skill in the art will appreciate that the specificity of a particular probe or primer increases with its length. Thus, for example, a primer comprising 20 consecutive nucleotides will anneal to a target with a higher specificity than a corresponding primer of only 15 nucleotides. Thus, in order to obtain greater specificity, probes and primers can be selected that comprise 20, 25, 30, 35, 40, 50 or more consecutive nucleotides.

Promoter: A promoter is an array of nucleic acid control sequences that directs transcription of a nucleic acid. A promoter includes necessary nucleic acid sequences near the start site of transcription, such as, in the case of a polymerase II type promoter, a TATA element. A promoter also optionally includes distal enhancers or repressor elements which can be located as much as several thousand base pairs from the start site of transcription. Both constitutive and inducible promoters are included (see e.g., Bitter et al., Methods in Enzymology 153:516-544, 1987).

Specific, non-limiting examples of promoters include promoters derived from the genome of mammalian cells (e.g., metallothionein promoter) or from mammalian viruses (e.g., the retrovirus long terminal repeat; the adenovirus late promoter; the vaccinia virus 7.5K promoter) may be used. Promoters produced by recombinant DNA or synthetic techniques may also be used. A polynucleotide can be inserted into an expression vector that contains a promoter sequence which facilitates the efficient transcription of the inserted genetic sequence of the host. The expression vector typically contains an origin of replication, a promoter, as well as specific nucleic acid sequences that allow phenotypic selection of the transformed cells

Purified: The term purified does not require absolute purity; rather, it is intended as a relative term. Thus, for example, a purified nucleic acid is one in which the nucleic acid is more enriched than the nucleic acid in its natural environment within a cell. Similarly, a purified peptide preparation is one in which the peptide or protein is more enriched than the peptide or protein is in its natural environment within a cell. Substantial purification denotes purification from other proteins or cellular components. In one embodiment, a preparation is purified such that the protein or peptide represents at least 50% (such as, but not limited to, 70%, 80%, 90%, 95%, 98% or 99%) of the total peptide or protein content of the preparation. The 68h05 polypeptides disclosed herein can be purified (and/or synthesized) by any of the means known in the art (see, e.g., Guide to Protein Purification, ed. Deutscher, Meth. Enzymol. 185, Academic Press, San Diego, 1990; and Scopes, Protein Purification: Principles and Practice, Springer Verlag, New York, 1982).

Recombinant: A recombinant nucleic acid is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques.

Selectively hybridize: Hybridization under moderately or highly stringent conditions that excludes non-related nucleotide sequences.

In nucleic acid hybridization reactions, the conditions used to achieve a particular level of stringency, will vary depending on the nature of the nucleic acids being hybridized. For example, the length, degree of complementarity, nucleotide sequence composition (e.g., GC v. AT content), and nucleic acid type (e.g., RNA versus DNA) of the hybridizing regions of the nucleic acids can be considered in selecting hybridization conditions. An additional consideration is whether one of the nucleic acids is immobilized, for example, on a filter.

A specific, non-limiting example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42° C. (moderate stringency conditions); and 0.1×SSC at about 68° C. (high stringency conditions). One of skill in the art can readily determine variations on these conditions (e.g., Molecular Cloning: A Laboratory Manual, 2nd ed., vol. 1-3, ed. Sambrook et al., Cold Spring Harbor Laboratory Press, Cold Spring Harbor, N.Y., 1989). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically.

Sequence identity: The similarity between amino acid sequences is expressed in terms of the similarity between the sequences, otherwise referred to as sequence identity. Sequence identity is frequently measured in terms of percentage identity (or similarity or homology); the higher the percentage, the more similar the two sequences are. Homologues or variants of a 68h05 polypeptide will possess a relatively high degree of sequence identity when aligned using standard methods.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. USA 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. USA 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

Homologs and variants of a 68h05 polypeptide are typically characterized by possession of at least 75%, for example at least 80%, sequence identity counted over the full length alignment with the amino acid sequence of 68h05 using the NCBI Blast 2.0, gapped blastp set to default parameters. For comparisons of amino acid sequences of greater than about 30 amino acids, the Blast 2 sequences function is employed using the default BLOSUM62 matrix set to default parameters (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95%, depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Specific binding agent: An agent that binds substantially only to a defined target. Thus a 68h05 specific binding agent is an agent that binds substantially to a 68h05 polypeptide. In one embodiment, the specific binding agent is a monoclonal or polyclonal antibody that specifically binds 68h05. In one specific, non-limiting example, the monoclonal or polyclonal antibody binds 68h05, but not NGEP.

Subject: Living multi-cellular vertebrate organisms, a category that includes both human and veterinary subjects, including human and non-human mammals.

T Cell: A white blood cell critical to the immune response. T cells include, but are not limited to, CD4⁺ T cells and CD8⁺ T cells. A CD4⁺ T lymphocyte is an immune cell that carries a marker on its surface known as “cluster of differentiation 4” (CD4). These cells, often called “helper” T cells, help orchestrate the immune response, including antibody responses as well as killer T cell responses. CD8⁺ T cells carry the “cluster of differentiation 8” (CD8) marker. In one embodiment, a CD8 T cell is a cytotoxic T lymphocytes. In another embodiment, a CD8 cell is a suppressor T cell.

Therapeutically active polypeptide: An agent, such as a 68h05 polypeptide that causes induction of an immune response, as measured by clinical response (for example increase in a population of immune cells, production of antibody that specifically binds 68h05, or measurable reduction of tumor burden). Therapeutically active molecules can also be made from nucleic acids. Examples of a nucleic acid based therapeutically active molecule is a nucleic acid sequence that encodes a 68h05 polypeptide, wherein the nucleic acid sequence is operably linked to a control element such as a promoter. Therapeutically active agents can also include organic or other chemical compounds that mimic the effects of 68h05.

The terms “therapeutically effective fragment of 68h05” or “therapeutically effective variant of 68h05” includes any fragment of 68h05, or variant of 68h05, that retains a function of 68h05, or retains an antigenic epitope of 68h05.

In one embodiment, a therapeutically effective amount of a fragment of 68h05 is an amount used to generate an immune response, or to treat breast or prostate cancer in a subject. Specific, non-limiting examples are the N-terminal half of 68h05 or the C-terminal half of 68h05. Treatment refers to a therapeutic intervention that ameliorates a sign or symptom of breast or prostate cancer, or a reduction in tumor burden.

Transduced: A transduced cell is a cell into which has been introduced a nucleic acid molecule by molecular biology techniques. As used herein, the term transduction encompasses all techniques by which a nucleic acid molecule might be introduced into such a cell, including transfection with viral vectors, transformation with plasmid vectors, and introduction of naked DNA by electroporation, lipofection, and particle gun acceleration.

Vector: A nucleic acid molecule as introduced into a host cell, thereby producing a transformed host cell. A vector may include nucleic acid sequences that permit it to replicate in a host cell, such as an origin of replication. A vector may also include one or more selectable marker genes and other genetic elements known in the art.

Unless otherwise explained, all technical and scientific terms used herein have the same meaning as commonly understood by one of ordinary skill in the art to which this disclosure belongs. The singular terms “a,” “an,” and “the” include plural referents unless the context clearly indicates otherwise. Similarly, the word “or” is intended to include “and” unless the context clearly indicates otherwise. “Comprise” means “include.” It is further to be understood that all base sizes or amino acid sizes, and all molecular weight or molecular mass values, given for nucleic acids or polypeptides are approximate, and are provided for description. Although methods and materials similar or equivalents to those described herein can be used in the practice or testing of the present disclosure, suitable methods and materials are described below. In case of conflict, the present specification, including explanations of terms, will control. In addition, the materials, methods, and examples are illustrative only and not intended to be limiting.

### 68h05 Polynucleotides and Polypeptides

68h05 polypeptides are disclosed herein. In one embodiment, a 68h05 polypeptide is 334 amino acids in length and has a sequence set forth as follows:

| | |
|---|---|
| MRGPSWSRPRPLLLLLLLLSPWPVWAQVSATASPSGSLGAPDCPEVC | (SEQ ID: NO 1) |
| TCVPGGLASCSALSLPAVPPGLSLRLRALLLDHNRVRALPPGAFAGA | |
| GALQRLDLRENGLHSVHVRAFWGLGALQLLDLSANQLEALAPGTFA | |
| PLRALRNLSLAGNRLARLEPAALGALPLLRSLSLQDNELAALAPGLL | |
| GRLPALDALHLRGNPWGCGCALRPLCAWLRRHPLPASEAETVLCV | |
| WPGRLTLSPLTAFSDAAFSHCAQPLALRDLAVVYTLGPASFLVSLAS | |
| CLALGSGLTACRARRRRLRTAALRPPRPPDPNPDPDPHGCASPADPG | |
| SPAAAAQA | |

In a second embodiment, a 68h05 polypeptide has a sequence at least 75%; 85%, 90%, 95%, 96%, 97%, 98% or 99% homologous to the amino acid sequence set forth in SEQ ID NO: 1. In one example, antibodies that bind to 68h05 bind to the homologous polypeptide. In another embodiment, a 68h05 polypeptide has a sequence as set forth in SEQ ID NO: 1 or is a conservative variant of SEQ ID NO: 1. In a further embodiment, a 68h05 polypeptide includes a sequence as set forth as SEQ ID NO: 1. Thus, polypeptides including a 68h05 and an additional polypeptide sequence, such as six histine residues, myc, and/or a signal sequence, are envisioned.

Fragments and variants of a 68h05 polypeptide can readily be prepared by one of skill in the art using molecular techniques. In one embodiment, a fragment of a 68h05 polypeptide includes at least 8, 10, 15, or 20 consecutive amino acids of a 68h05 polypeptide.

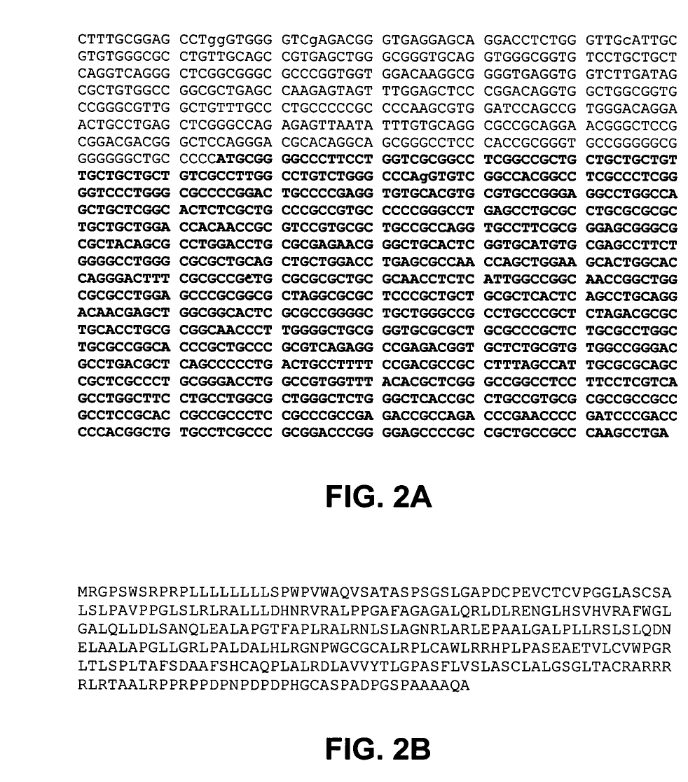

68h05 polypeptide sequences include, but are not limited to, the following 68h05 fragments (see FIG. 2):

| Amino Acid Position | |
|---|---|
| (see SEQ ID NO: 2) | Structural Region |
| 1-26 | Signal peptide |
| 45-75 | N-terminal leucine rich |
| | region (LRR) |
| 76-93 | LRR |
| 99-117 | LRR |
| 118-141 | LRR |
| 142-165 | LRR |
| 166-189 | LRR |
| 201-254 | C-terminal LRR |
| 265-287 | Transmembrane domain |

In another embodiment, a 68h05 polypeptide is a fragment of a full-length 68h05 polypeptide that includes a specific antigenic epitope found on full-length 68h05. An antibody or an MHC molecule can specifically bind the antigenic epitope. Thus, an antibody that binds full length 68h05 (SEQ ID NO: 1) binds the 68h05 fragment. These fragments include, but are not limited to, a polypeptide including at least 8 consecutive amino acids of the cytoplasmic region of 68h05, such as at least 8 (e.g. 10, 15, 20, 25, etc.) consecutive amino acids of the sequence shown as amino acids 45-75 of SEQ ID NO: 1, amino acids 76-93 of SEQ ID NO: 1, amino acids 99-117 of SEQ ID NO: 1, or amino acids 142-165of SEQ ID NO: 1. These fragments also include, a polypeptide including at least 8 consecutive amino acids of the external (cell-surface) region of 68h05, such as, but not limited to, 8 consecutive amino acids of the sequence shown as amino acids 27-264 of SEQ ID NO: 1, amino acids 201-254 of SEQ ID NO: 1, or amino acids 265-334 of SEQ ID NO: 1. In another embodiment, the fragment is a peptide, such as a 9- or a 10-mer that specifically binds an MHC molecule.

Suitable polypeptides include amino acids 1-64 of SEQ ID NO: 1, amino acids 173-190 of SEQ ID NO: 1, amino acids 215-228 of SEQ ID NO: 1 and amino acids 261-334 of SEQ ID NO: 1, or at least eight consecutive amino acids thereof. In one example, these polypeptides do not include the full length 68h05 polypeptide (SEQ ID NO: 1). These polypeptides include, but not limited to nine or ten consecutive amino acids of amino acids 1-64 of SEQ ID NO: 1, amino acids 173-190 of SEQ ID NO: 1, amino acids 215-228 of SEQ ID NO: 1 and amino acids 261 -334 of SEQ ID NO: 1. In one specific non-limiting example, the polypeptide comprises at least eight consecutive amino acids of CRARRRRLRTAALRPPRPPDPNPDPDPHG (amino acids 290-318 of SEQ ID NO: 1), such as nine or ten consecutive amino acids of this sequence. In another example, the polypeptide is CRARRRRLRTAALRPPRPPDPNPDPDPHG (amino acids 290-318 of SEQ ID NO: 1).

The presentation of peptides by MHC Class I molecules involves the cleavage of an endogenously produced protein into peptides by the proteasome, its processing through the ER and Golgi apparatus, its binding to the cleft in an MHC Class I molecule through the anchor residues of the peptide and ultimate presentation on the cell surface. Depending upon the particular anchor residues, among other things, certain peptides may bind more tightly to a particular HLA molecules than others. Peptides that bind well are referred to as “dominant” epitopes, while those that bind less well are termed “subdominant” or “cryptic” epitopes. Dominant epitopes of either self proteins or foreign proteins evoke strong tolerance or immune responses. Subdominant or cryptic epitopes generate weak responses or no responses at all. Without being by theory, tighter binding by dominant epitopes to HLA molecules results in their denser presentation on the cell surface, greater opportunity to react with immune cells and greater likelihood of eliciting an immune response or tolerance. MHC Class I molecules present epitopes from endogenous proteins for presentation to T_{C} cells. HLA A, HLA B and HLA C molecules bind peptides of about 8 to 10 amino acids in length that have particular anchoring residues. The anchoring residues recognized by an HLA Class I molecule depend upon the particular allelic form of the HLA molecule. A CD8⁺ T cell bears T cell receptors that recognize a specific epitope when presented by a particular HLA molecule on a cell. When a T_{C} cell that has been stimulated by an antigen presenting cell to become a cytotoxic T lymphocyte contacts a cell that bears such an HLA-peptide complex, the CTL forms a conjugate with the cell and destroys it. Programs are available on the internet for the prediction of epitopes that bind MHC.

For example, an HLA binding motif program on the internet (Bioinformatics and Molecular Analysis Section-BIMAS) predicts the following 9-mers of SEQ ID NO: 1 that will bind HLA2-01:
LLLLSPWPV, SEQ ID NO: 3, starting at position 16 of SEQ ID NO: 1,
TLGPASFLV, SEQ ID NO: 4, which starts at position 267 of SEQ ID NO: 1
ALLLDHNRV, SEQ ID NO: 5, which starts at position 75 of SEQ ID NO: 1
GLLGRLPAL, SEQ ID NO: 6, which starts at position 185 of SEQ ID NO: 1
FLVSLASCL, SEQ ID NO: 7, which starts at position 273 of SEQ ID NO: 1

One skilled in the art, given the disclosure herein, can purify a 68h05 polypeptide, or 68h05 9-mers, or other fragments, using standard techniques for protein purification. The substantially pure polypeptide will yield a single major band on a non-reducing polyacrylamide gel. The purity of the 68h05 polypeptide can also be determined by amino-terminal amino acid sequence analysis.

Minor modifications of the 68h05 polypeptide primary amino acid sequences may result in peptides which have substantially equivalent activity as compared to the unmodified counterpart polypeptide described herein. Such modifications may be deliberate, as by site-directed mutagenesis, or may be spontaneous. All of the polypeptides produced by these modifications are included herein.

Thus, a specific, non-limiting example of a 68h05 polypeptide is a conservative variant of 68h05. A table of conservative substitutions is provided herein. Substitutions of the amino acids sequence shown in SEQ ID NO: 1 can be made based on this table. Thus, one non-limiting example of a conservative variant is substitution of amino acid one (Met) of SEQ ID NO: 1 with an arginine residue. Similarly, another non-limiting example is substitution of amino acid 2 (Arg) of SEQ ID NO: 1 with a lysine residue. Using the sequence provided as SEQ ID NO: 1, and the description of conservative amino acid substitutions provided, one of skill in the art can readily ascertain sequences of conservative variants. In several embodiments, a conservative variant includes at most one, at most two, at most five, at most ten, or at most fifteen conservative substitutions of the sequence shown in SEQ ID NO: 1. Generally, a conservative variant will bind to antibodies that immunoreact with (specifically bind to) a polypeptide having a sequence set forth as SEQ ID NO:1.

One of skill in the art can readily produce fusion proteins including a 68h05 polypeptide and a second polypeptide of interest. Optionally, a linker can be included between the 68h05 polypeptide and the second polypeptide of interest. Fusion proteins include, but are not limited to, a polypeptide including a 68h05 polypeptide and a marker protein. In one embodiment, the marker protein can be used to identify or purify a 68h05 polypeptide. Exemplary fusion proteins included, but are not limited, to green fluorescent protein, six histidine residues, or myc and a 68h05 polypeptide.

Polynucleotides encoding a 68h05 polypeptide are also provided, and are termed 68h05 polynucleotides. These polynucleotides include DNA, cDNA and RNA sequences which encode 68h05. It is understood that all polynucleotides encoding a 68h05 polypeptide are also included herein, as long as they encode a polypeptide with the recognized activity, such as the binding to an antibody that recognizes a 68h05 polypeptide. The polynucleotides of this disclosure include sequences that are degenerate as a result of the genetic code. There are 20 natural amino acids, most of which are specified by more than one codon. Therefore, all degenerate nucleotide sequences are included in the invention as long as the amino acid sequence of the 68h05 polypeptide encoded by the nucleotide sequence is functionally unchanged. One specific, non-limiting example of a polynucleotide. encoding 68h05 is:

| | | | | | | |
|---|---|---|---|---|---|---|
| CTTTGCGGAG | CCTggGTGGG | GTCgAGACGG | GTGAGGAGCA | GGACCTCTGG | GTTGcATTGC | (SEQ ID NO: 2) |
| GTGTGGGCGC | CTGTTGCAGC | CGTGAGCTGG | GCGGGTGCAG | GTGGGCGGTG | TCCTGCTGCT | |
| CAGGTCAGGG | CTCGGCGGGC | GCCCGGTGGT | GGACAAGGCG | GGGTGAGGTG | GTCTTGATAG | |
| CGCTGTGGCC | GGCGCTGAGC | CAAGAGTAGT | TTGGAGCTCC | CGGACAGGTG | GCTGGCGGTG | |
| CCGGGCGTTG | GCTGTTTGCC | CTGCCCCCGC | CCCAAGCGTG | GATCCAGCCG | TGGGACAGGA | |
| ACTGCCTGAG | CTCGGGCCAG | AGAGTTAATA | TTTGTGCAGG | CGCCGCAGGA | ACGGGCTCCG | |
| CGGACGACGG | GCTCCAGGGA | CGCACAGGCA | GCGGGCCTCC | CACCGCGGGT | GCCGGGGGCG | |
| GGGGGGCTGC | CCCCATGCGG | GGCCCTTCCT | GGTCGCGGCC | TCGGCCGCTG | CTGCTGCTGT | |
| TGCTGCTGCT | GTCGCCTTGG | CCTGTCTGGG | CCCAgGTGTC | GGCCACGGCC | TCGCCCTCGG | |
| GGTCCCTGGG | CGCCCCGGAC | TGCCCCGAGG | TGTGCACGTG | CGTGCCGGGA | GGCCTGGCCA | |
| GCTGCTCGGC | ACTCTCGCTG | CCCGCCGTGC | CCCCGGGCCT | GAGCCTGCGC | CTGCGCGCGC | |
| TGCTGCTGGA | CCACAACCGC | GTCCGTGCGC | TGCCGCCAGG | TGCCTTCGCG | GGAGCGGGCG | |
| CGCTACAGCG | CCTGGACCTG | CGCGAGAACG | GGCTGCACTC | GGTGCATGTG | CGAGCCTTCT | |
| GGGGCCTGGG | CGCGCTGCAG | CTGCTGGACC | TGAGCGCCAA | CCAGCTGGAA | GCACTGGCAC | |
| CAGGGACTTT | CGCGCCGCTG | CGCGCGCTGC | GCAACCTCTC | ATTGGCCGGC | AACCGGCTGG | |
| CGCGCCTGGA | GCCCGCGGCG | CTAGGCGCGC | TCCCGCTGCT | GCGCTCACTC | AGCCTGCAGG | |
| ACAACGAGCT | GGCGGCACTC | GCGCCGGGGC | TGCTGGGCCG | CCTGCCCGCT | CTAGACGCGC | |
| TGCACCTGCG | CGGCAACCCT | TGGGGCTGCG | GGTGCGCGCT | GCGCCCGCTC | TGCGCCTGGC | |
| TGCGCCGGCA | CCCGCTGCCC | GCGTCAGAGG | CCGAGACGGT | GCTCTGCGTG | TGGCCGGGAC | |
| GCCTGACGCT | CAGCCCCCTG | ACTGCCTTTT | CCGACGCCGC | CTTTAGCCAT | TGCGCGCAGC | |
| CGCTCGCCCT | GCGGGACCTG | GCCGTGGTTT | ACACGCTCGG | GCCGGCCTCC | TTCCTCGTCA | |
| GCCTGGCTTC | CTGCCTGGCG | CTGGGCTCTG | GGCTCACCGC | CTGCCGTGCG | CGCCGCCGCC | |
| GCCTCCGCAC | CGCCGCCCTC | CGCCCGCCGA | GACCGCCAGA | CCCGAACCCC | GATCCCGACC | |
| CCCACGGCTG | TGCCTCGCCC | GCGGACCCGG | GGAGCCCCGC | CGCTGCCGCC | CAAGCCTGA | |

The open reading frame is shown in bold in the above sequence, and in FIG. 2. Thus, another example of a nucleic acid encoding 68h05 is a nucleic acid including only the nucleotides shown in bold in the above sequence.

Another specific non-limiting example of a polynucleotide encoding 68h05 is a polynucleotide having at least 75%, 85%, 90%, 95%, or 99% homologous to SEQ ID NO: 2 that encodes a polypeptide having an antigenic epitope or function of 68h05. Yet another specific non-limiting example of a polynucleotide encoding 68h05 is a polynucleotide that encodes a polypeptide that is specifically bound by an antibody that specifically binds SEQ ID NO: 1.

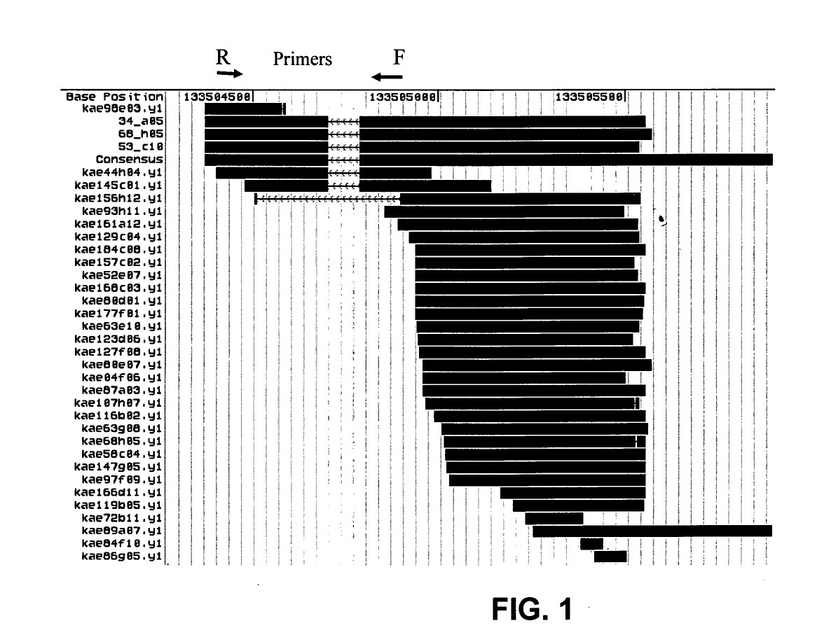

Primers, such as PCR primers can readily be prepared from a 68h05 polynucleotide. In one embodiment, the primers include at least ten, at least 15, 16, 17, 18, 18, or 20 consecutive nucleotides of SEQ ID NO: 2. Exemplary primers are disclosed herein, such as the primers shown in FIG. 1 and discussed in the Examples section. Also included are fragments of the above-described nucleic acid sequences that are at least 15 bases in length, which is sufficient to permit the fragment to selectively hybridize to DNA that encodes the disclosed 68h05 polypeptide (e.g. a polynucleotide that encodes SEQ ID NO: 1) under stringent hybridization conditions. The term “selectively hybridize” refers to hybridization under moderately or highly stringent conditions, which excludes non-related nucleotide sequences.

A nucleic acid encoding 68h05 can be cloned or amplified by in vitro methods, such as the polymerase chain reaction (PCR), the ligase chain reaction (LCR), the transcription-based amplification system (TAS), the self-sustained sequence replication system (3SR) and the Qβ replicase amplification system (QB). For example, a polynucleotide encoding the protein can be isolated by polymerase chain reaction of cDNA using primers based on the DNA sequence of the molecule. A wide variety of cloning and in vitro amplification methodologies are well known to persons skilled in the art. PCR methods are described in, for example, U.S. Pat. No. 4,683,195; Mullis et al., Cold Spring Harbor Symp. Quant. Biol. 51:263, 1987; and Erlich, ed., PCR Technology, (Stockton Press, NY, 1989). Polynucleotides also can be isolated by screening genomic or cDNA libraries with probes selected from the sequences of the desired polynucleotide under stringent hybridization conditions.

The 68h05 polynucleotides include a recombinant DNA which is incorporated into a vector; into an autonomously replicating plasmid or virus; or into the genomic DNA of a prokaryote or eukaryote, or which exists as a separate molecule (e.g., a cDNA) independent of other sequences. The nucleotides of the invention can be ribonucleotides, deoxyribonucleotides, or modified forms of either nucleotide. The term includes single and double forms of DNA. The 68h05 polynucleotide sequence disclosed herein include, but are not limited to, SEQ ID NO: 2, degenerate variants of SEQ ID NO: 2, sequences that encode conservative variations of 68h05 polypeptide, and sequences that encode fragments of SEQ ID NO: 2.

DNA sequences encoding 68h05 polypeptide can be expressed in vitro by DNA transfer into a suitable host cell. The cell may be prokaryotic or eukaryotic. The term also includes any progeny of the subject host cell. It is understood that all progeny may not be identical to the parental cell since there may be mutations that occur during replication. Methods of stable transfer, meaning that the foreign DNA is continuously maintained in the host, are known in the art.

68h05 polynucleotide sequences can be operatively linked to expression control sequences. An expression control sequence operatively linked to a coding sequence is ligated such that expression of the coding sequence is achieved under conditions compatible with the expression control sequences. The expression control sequences include, but are not limited to, appropriate promoters, enhancers, transcription terminators, a start codon (i.e., ATG) in front of a protein-encoding gene, splicing signal for introns, maintenance of the correct reading frame of that gene to permit proper translation of mRNA, and stop codons.

The polynucleotide sequences encoding 68h05 may be inserted into an expression vector including, but not limited to, a plasmid, virus or other vehicle that can be manipulated to allow insertion or incorporation of sequences and can be expressed in either prokaryotes or eukaryotes. Hosts can include microbial, yeast, insect and mammalian organisms. Methods of expressing DNA sequences having eukaryotic or viral sequences in prokaryotes are well known in the art. Biologically functional viral and plasmid DNA vectors capable of expression and replication in a host are known in the art.

Transformation of a host cell with recombinant DNA may be carried out by conventional techniques as are well known to those skilled in the art. Where the host is prokaryotic, such as E. coli, competent cells which are capable of DNA uptake can be prepared from cells harvested after exponential growth phase and subsequently treated by the CaCl₂ method using procedures well known in the art. Alternatively, MgCl₂ or RbCl can be used. Transformation can also be performed after forming a protoplast of the host cell if desired, or by electroporation.

When the host is a eukaryote, such methods of transfection of DNA as calcium phosphate coprecipitates, conventional mechanical procedures such as microinjection, electroporation, insertion of a plasmid encased in liposomes, or virus vectors may be used. Eukaryotic cells can also be cotransformed with 68h05 polynucleotide sequences, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Eukaryotic Viral Vectors, Cold Spring Harbor Laboratory, Gluzman ed., 1982).

Isolation and purification of recombinantly expressed polypeptides may be carried out by conventional means including preparative chromatography and immunological separations.

### Antibodies

A 68h05 polypeptide or a fragment or conservative variant thereof can be used to produce antibodies which are immunoreactive or bind to an epitope of 68h05. Polyclonal antibodies, antibodies which consist essentially of pooled monoclonal antibodies with different epitope specificities, as well as distinct monoclonal antibody preparations are included.

An antibody can bind one of the following amino acids fragments of a 68h05 polypeptide:

| Amino Acid Position | |
|---|---|
| (see SEQ ID NO: 2) | Structural Region |
| 1-26 | Signal peptide |
| 45-75 | N-terminal leucine rich |
| | region (LRR) |
| 76-93 | LRR |
| 99-117 | LRR |
| 118-141 | LRR |
| 142-165 | LRR |
| 166-189 | LRR |
| 201-254 | C-terminal LRR |
| 265-287 | Transmembrane domain |

In one embodiment, an antibody can specifically bind any antigenic epitope of 68h05, and can also bind full-length 68h05. The antibody can bind 68h05 fragments including, but are not limited to, a polypeptide including at least 8 consecutive amino acids of the cytoplasmic region of 68h05, such as at least 8 (e.g. 10, 15, 20, 25, etc.) consecutive amino acids of the sequence shown as amino acids 45-75 of SEQ ID NO: 1, amino acids 76-93 of SEQ ID NO: 1, amino acids 99-117 of SEQ ID NO: 1, or amino acids 142-165of SEQ ID NO: 1, amino acids 27-264 of SEQ ID NO: 1, amino acids 201-254 of SEQ ID NO: 1, or amino acids 265-334 of SEQ ID NO: 1.

In several embodiments, the antibody binds amino acids 1-64 of SEQ ID NO: 1, amino acids 173-190 of SEQ ID NO: 1, amino acids 215-228 of SEQ ID NO: 1 and amino acids 261-334 of SEQ ID NO: 1, or at least eight consecutive amino acids thereof. These polypeptides include, but not limited to nine or ten consecutive amino acids of amino acids 1-64 of SEQ ID NO: 1, amino acids 173-190 of SEQ ID NO: 1, amino acids 215-228 of SEQ ID NO: 1 and amino acids 261-334 of SEQ ID NO: 1. In one specific non-limiting example, antibody binds at least eight consecutive amino acids of CRARRRRLRTAALRPPRPPDPNPDPDPHG (amino acids 290-318 of SEQ ID NO: 1).

The preparation of polyclonal antibodies is well known to those skilled in the art. See, for example, Green et al., “Production of Polyclonal Antisera, in: Immunochemical Protocols pages 1-5, Manson, ed., Humana Press, 1992; Coligan et al., “Production of Polyclonal Antisera in Rabbits, Rats, Mice and Hamsters, in: Current Protocols in Immunology, section 2.4.1, 1992. Exemplary methods and antibodies are disclosed in the examples section below.

The preparation of monoclonal antibodies likewise is conventional. See, for example, Kohler & Milstein, Nature 256:495, 1975; Coligan et al., sections 2.5.1-2.6.7; and Harlow et al., in: Antibodies: a Laboratory Manual, page 726, Cold Spring Harbor Pub., 1988. Briefly, monoclonal antibodies can be obtained by injecting mice with a composition comprising an antigen, verifying the presence of antibody production by removing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, e.g., Coligan et al., sections 2.7.1-2.7.12 and sections 2.9.1-2.9.3; Barnes et al., “Purification of Immunoglobulin G (IgG),” in: Methods in Molecular Biology, Vol. 10, pages 79-104, Humana Press, 1992.

Methods of in vitro and in vivo multiplication of monoclonal antibodies are well known to those skilled in the art. Multiplication in vitro may be carried out in suitable culture media such as Dulbecco's Modified Eagle Medium or RPMI 1640 medium, optionally supplemented by a mammalian serum such as fetal calf serum or trace elements and growth-sustaining supplements such as normal mouse peritoneal exudate cells, spleen cells, thymocytes or bone marrow macrophages. Production in vitro provides relatively pure antibody preparations and allows scale-up to yield large amounts of the desired antibodies. Large-scale hybridoma cultivation can be carried out by homogenous suspension culture in an airlift reactor, in a continuous stirrer reactor, or in immobilized or entrapped cell culture. Multiplication in vivo may be carried out by injecting cell clones into mammals histocompatible with the parent cells, e.g., syngeneic mice, to cause growth of antibody-producing tumors. Optionally, the animals are primed with a hydrocarbon, especially oils such as pristane (tetramethylpentadecane) prior to injection. After one to three weeks, the desired monoclonal antibody is recovered from the body fluid of the animal.

Antibodies can also be derived from a subhuman primate antibody. General techniques for raising therapeutically useful antibodies in baboons can be found, for example, in WO 91/11465, 1991, and Losman et al., Int. J. Cancer 46:310, 1990.

Alternatively, an antibody that specifically binds a 68h05 polypeptide can be derived from a humanized monoclonal antibody. Humanized monoclonal antibodies are produced by transferring mouse complementarity determining regions from heavy and light variable chains of the mouse immunoglobulin into a human variable domain, and then substituting human residues in the framework regions of the murine counterparts. The use of antibody components derived from humanized monoclonal antibodies obviates potential problems associated with the immunogenicity of murine constant regions. General techniques for cloning murine immunoglobulin variable domains are described, for example, by Orlandi et al., Proc. Nat'l Acad. Sci. USA 86:3833, 1989. Techniques for producing humanized monoclonal antibodies are described, for example, by Jones et al., Nature 321:522, 1986; Riechmann et al., Nature 332:323, 1988; Verhoeyen et al., Science 239:1534, 1988; Carter et al., Proc. Nat'l Acad. Sci. USA 89:4285, 1992; Sandhu, Crit. Rev. Biotech. 12:437, 1992; and Singer et al., J. Immunol. 150:2844, 1993.

Antibodies can be derived from human antibody fragments isolated from a combinatorial immunoglobulin library. See, for example, Barbas et al., in: Methods: a Companion to Methods in Enzymology, Vol. 2, page 119, 1991; Winter et al., Ann. Rev. Immunol. 12:433, 1994. Cloning and expression vectors that are useful for producing a human immunoglobulin phage library can be obtained, for example, from STRATAGENE Cloning Systems (La Jolla, Calif.).

In addition, antibodies can be derived from a human monoclonal antibody. Such antibodies are obtained from transgenic mice that have been “engineered” to produce specific human antibodies in response to antigenic challenge. In this technique, elements of the human heavy and light chain loci are introduced into strains of mice derived from embryonic stem cell lines that contain targeted disruptions of the endogenous heavy and light chain loci. The transgenic mice can synthesize human antibodies specific for human antigens, and the mice can be used to produce human antibody-secreting hybridomas. Methods for obtaining human antibodies from transgenic mice are described by Green et al., Nature Genet. 7:13, 1994; Lonberg et al., Nature 368:856, 1994; and Taylor et al., Int. Immunol. 6:579, 1994.

Antibodies include intact molecules as well as fragments thereof, such as Fab, F(ab′)₂, and Fv which are capable of binding the epitopic determinant. These antibody fragments retain some ability to selectively bind with their antigen or receptor and are defined as follows:
(1) Fab, the fragment which contains a monovalent antigen-binding fragment of an antibody molecule, can be produced by digestion of whole antibody with the enzyme papain to yield an intact light chain and a portion of one heavy chain;
(2) Fab′, the fragment of an antibody molecule can be obtained by treating whole antibody with pepsin, followed by reduction, to yield an intact light chain and a portion of the heavy chain; two Fab′ fragments are obtained per antibody molecule;
(3) (Fab′)₂, the fragment of the antibody that can be obtained by treating whole antibody with the enzyme pepsin without subsequent reduction; F(ab′)₂ is a dimer of two Fab′ fragments held together by two disulfide bonds;
(4) Fv, defined as a genetically engineered fragment containing the variable region of the light chain and the variable region of the heavy chain expressed as two chains; and
(5) Single chain antibody (SCA), defined as a genetically engineered molecule containing the variable region of the light chain, the variable region of the heavy chain, linked by a suitable polypeptide linker as a genetically fused single chain molecule.

Methods of making these fragments are known in the art. (See for example, Harlow and Lane, Antibodies: A Laboratory Manual, Cold Spring Harbor Laboratory, New York, 1988). An epitope is any antigenic determinant on an antigen to which the paratope of an antibody binds. Epitopic determinants usually consist of chemically active surface groupings of molecules such as amino acids or sugar side chains, and usually have specific three-dimensional structural characteristics, as well as specific charge characteristics.

Antibody fragments can be prepared by proteolytic hydrolysis of the antibody or by expression in E. coli of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly (see U.S. Pat. No. 4,036,945 and U.S. Pat. No. 4,331,647, and references contained therein; Nisonhoff et al., Arch. Biochem. Biophys. 89:230, 1960; Porter, Biochem. J. 73:119, 1959; Edelman et al., Methods in Enzymology, Vol. 1, page 422, Academic Press, 1967; and Coligan et al., supra at sections 2.8.1-2.8.10 and 2.10.1-2.10.4).

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

For example, Fv fragments comprise an association of V_{H} and V_{L} chains. This association may be noncovalent (Inbar et al., Proc. Nat'l Acad. Sci. USA 69:2659, 1972). Alternatively, the variable chains can be linked by an intermolecular disulfide bond or cross-linked by chemicals such as glutaraldehyde. See, e.g., Sandhu, supra. Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are known in the art (see Whitlow et al., Methods: a Companion to Methods in Enzymology, Vol. 2, page 97, 1991; Bird et al., Science 242:423, 1988; U.S. Pat. No. 4,946,778; Pack et al., Bio/Technology 11:1271, 1993; and Sandhu, supra).

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells (Larrick et al., Methods: a Companion to Methods in Enzymology, Vol. 2, page 106, 1991).

Antibodies can be prepared using an intact polypeptide or fragments containing small peptides of interest as the immunizing antigen. The polypeptide or a peptide used to immunize an animal can be derived from substantially purified polypeptide produced in host cells, in vitro translated cDNA, or chemical synthesis which can be conjugated to a carrier protein, if desired. Such commonly used carriers which are chemically coupled to the peptide include keyhole limpet hemocyanin (KLH), thyroglobulin, bovine serum albumin (BSA), and tetanus toxoid. The coupled peptide is then used to immunize the animal (e.g., a mouse, a rat, or a rabbit).

Polyclonal or monoclonal antibodies can be further purified, for example, by binding to and elution from a matrix to which the polypeptide or a peptide to which the antibodies were raised is bound. Those of skill in the art will know of various techniques common in the immunology arts for purification and/or concentration of polyclonal antibodies, as well as monoclonal antibodies (See for example, Coligan et al., Unit 9, Current Protocols in Immunology, Wiley Interscience, 1991).

It is also possible to use the anti-idiotype technology to produce monoclonal antibodies which mimic an epitope. For example, an anti-idiotypic monoclonal antibody made to a first monoclonal antibody will have a binding domain in the hypervariable region that is the “image” of the epitope bound by the first mono-clonal antibody.

Binding affinity for a target antigen is typically measured or determined by standard antibody-antigen assays, such as competitive assays, saturation assays, or immunoassays such as ELISA or RIA. Such assays can be used to determine the dissociation constant of the antibody. The phrase “dissociation constant” refers to the affinity of an antibody for an antigen. Specificity of binding between an antibody and an antigen exists if the dissociation constant (K_{D}=1/K, where K is the affinity constant) of the antibody is, for example <1 μM, <100 μM, or <0.1 μM. Antibody molecules will typically have a K_{D} in the lower ranges. K_{D}=[Ab-Ag]/[Ab][Ag] where [Ab] is the concentration at equilibrium of the antibody, [Ag] is the concentration at equilibrium of the antigen and [Ab-Ag] is the concentration at equilibrium of the antibody-antigen complex. Typically, the binding interactions between antigen and antibody include reversible noncovalent associations such as electrostatic attraction, Van der Waals forces and hydrogen bonds.

Effector molecules, e.g., therapeutic, diagnostic, or detection moieties can be linked to an antibody that specifically binds 68h05, using any number of means known to those of skill in the art. Exemplary effector molecules include, but not limited to, a radiolabels, fluorescent markers, or toxins (e.g. Pseudomonas exotoxin (PE), see “Monoclonal Antibody-Toxin Conjugates: Aiming the Magic Bullet,” Thorpe et al., “Monoclonal Antibodies in Clinical Medicine”, Academic Press, pp. 168-190, 1982; Waldmann, Science, 252: 1657, 1991; U.S. Pat. No. 4,545,985 and U.S. Pat. No. 4,894,443, for a discussion of toxins and conjugation). Both covalent and noncovalent attachment means may be used. The procedure for attaching an effector molecule to an antibody varies according to the chemical structure of the effector. Polypeptides typically contain variety of functional groups; e.g., carboxylic acid (COOH), free amine (—NH₂) or sulfhydryl (—SH) groups, which are available for reaction with a suitable functional group on an antibody to result in the binding of the effector molecule. Alternatively, the antibody is derivatized to expose or attach additional reactive functional groups. The derivatization may involve attachment of any of a number of linker molecules such as those available from Pierce Chemical Company, Rockford, Ill. The linker can be any molecule used to join the antibody to the effector molecule. The linker is capable of forming covalent bonds to both the antibody and to the effector molecule. Suitable linkers are well known to those of skill in the art and include, but are not limited to, straight or branched-chain carbon linkers, heterocyclic carbon linkers, or peptide linkers. Where the antibody and the effector molecule are polypeptides, the linkers may be joined to the constituent amino acids through their side groups (for example, through a disulfide linkage to cysteine) or to the alpha carbon amino and carboxyl groups of the terminal amino acids.

In some circumstances, it is desirable to free the effector molecule from the antibody when the immunoconjugate has reached its target site. Therefore, in these circumstances, immunoconjugates will comprise linkages that are cleavable in the vicinity of the target site. Cleavage of the linker to release the effector molecule from the antibody may be prompted by enzymatic activity or conditions to which the immunoconjugate is subjected either inside the target cell or in the vicinity of the target site. When the target site is a tumor, a linker which is cleavable under conditions present at the tumor site (e.g. when exposed to tumor-associated enzymes or acidic pH) can be used.

In view of the large number of methods that have been reported for attaching a variety of radiodiagnostic compounds, radiotherapeutic compounds, label (e.g. enzymes or fluorescent molecules) drugs, toxins, and other agents to antibodies one skilled in the art will be able to determine a suitable method for attaching a given agent to an antibody or other polypeptide.

### Therapeutic Methods and Pharmaceutical Compositions

Methods for treating a subject, such as a subject with breast or prostate cancer, by administering a therapeutically effective amount of a pharmaceutical composition are disclosed herein. For example, 68h05 polypeptide can be administered to a subject in order to generate an immune response. In one embodiment, a therapeutically effective amount of a 68h05 polypeptide is administered to a subject to treat breast or prostate cancer.

In exemplary applications, compositions are administered to a patient suffering from a disease, such as breast or prostate cancer, in an amount sufficient to raise an immune response to 68h05-expressing cells. Administration induces a sufficient immune response to slow the proliferation of such cells or to inhibit their growth. Amounts effective for this use will depend upon the severity of the disease, the general state of the patient's health, and the robustness of the patient's immune system. A therapeutically effective amount of the compound is that which provides either subjective relief of a symptom(s) or an objectively identifiable improvement as noted by the clinician or other qualified observer.

A 68h05 polypeptide can be administered by any means known to one of skill in the art (see Banja, A., Parenteral Controlled Delivery of Therapeutic Peptides and Proteins, in Therapeutic Peptides and Proteins, Technomic Publishing Co., Inc., Lancaster, Pa., 1995) such as by intramuscular, subcutaneous, or intravenous injection, but even oral, nasal, or anal administration is contemplated. In one embodiment, administration is by subcutaneous or intramuscular injection. To extend the time during which the peptide or protein is available to stimulate a response, the peptide or protein can be provided as an implant, an oily injection, or as a particulate system. The particulate system can be a microparticle, a microcapsule, a microsphere, a nanocapsule, or similar particle. (see, e.g., Banja, supra). A particulate carrier based on a synthetic polymer has been shown to act as an adjuvant to enhance the immune response, in addition to providing a controlled release. Aluminum salts may also be used as adjuvants to produce a humoral immune response. Thus, in one embodiment, a 68h05 polypeptide is administered in a manner to induce a humoral response.

In another embodiment, a 68h05 polypeptide is administered in a manner to direct the immune response to a cellular response (that is, a CTL response), rather than a humoral (antibody) response. A number of means for inducing cellular responses, both in vitro and in vivo, are known. Lipids have been identified as agents capable of assisting in priming CTL in vivo against various antigens. For example, as described in U.S. Pat. No. 5,662,907, palmitic acid residues can be attached to the alpha and epsilon amino groups of a lysine residue and then linked (e.g., via one or more linking residues, such as glycine, glycine-glycine, serine, serine-serine, or the like) to an immunogenic peptide. The lipidated peptide can then be injected directly in a micellar form, incorporated in a liposome, or emulsified in an adjuvant. As another example, E. coli lipoproteins, such as tripalmitoyl-S-glycerylcysteinlyseryl-serine can be used to prime tumor specific CTL when covalently attached to an appropriate peptide (see, Deres et al., Nature 342:561, 1989). Further, as the induction of neutralizing antibodies can also be primed with the same molecule conjugated to a peptide which displays an appropriate epitope, the two compositions can be combined to elicit both humoral and cell-mediated responses where that is deemed desirable.

In yet another embodiment, to induce a CTL response to an immunogenic 68h05 polypeptide or fragment thereof, a MHC Class II-restricted T-helper epitope is added to the CTL antigenic peptide to induce T-helper cells to secrete cytokines in the microenvironment to activate CTL precursor cells. The technique further involves adding short lipid molecules to retain the construct at the site of the injection for several days to localize the antigen at the site of the injection and enhance its proximity to dendritic cells or other “professional” antigen presenting cells over a period of time (see Chesnut et al., “Design and Testing of Peptide-Based Cytotoxic T-Cell-Mediated Immunotherapeutics to Treat Infectious Diseases and Cancer,” in Powell et al., eds., Vaccine Design, the Subunit and Adjuvant Approach, Plenum Press, New York, 1995).

A pharmaceutical composition including a 68h05 polypeptide is thus provided. In one embodiment, the 68h05 polypeptide, or fragment thereof, is mixed with an adjuvant containing two or more of a stabilizing detergent, a micelle-forming agent, and an oil. Suitable stabilizing detergents, micelle-forming agents, and oils are detailed in U.S. Pat. No. 5, 585,103; U.S. Pat. No. 5,709,860; U.S. Pat. No. 5,270,202; and U.S. Pat. No. 5,695,770, all of which are incorporated by reference. A stabilizing detergent is any detergent that allows the components of the emulsion to remain as a stable emulsion. Such detergents include polysorbate, 80 (TWEEN) (Sorbitan-mono-9-octadecenoate-poly(oxy-1,2-ethanediyl; manufactured by ICI Americas, Wilmington, Del.), TWEEN 40™, TWEEN 20™, TWEEN 60™, Zwittergent™ 3-12, TEEPOL HB7™, and SPAN 85™. These detergents are usually provided in an amount of approximately 0.05 to 0.5%, preferably at about 0.2%. A micelle forming agent is an agent which is able to stabilize the emulsion formed with the other components such that a micelle-like structure is formed. Such agents generally cause some irritation at the site of injection in order to recruit macrophages to enhance the cellular response. Examples of such agents include polymer surfactants described by BASF Wyandotte publications, e.g., Schmolka, J. Am. Oil. Chem. Soc. 54:110, 1977, and Hunter et al., J. Immunol 129:1244, 1981, PLURONIC™ L62LF, L101, and L64, PEG1000, and TETRONIC™ 1501, 150R1, 701, 901, 1301, and 130R1. The chemical structures of such agents are well known in the art. In one embodiment, the agent is chosen to have a hydrophile-lipophile balance (HLB) of between 0 and 2, as defined by Hunter and Bennett, J. Immun. 133:3167, 1984. The agent can be provided in an effective amount, for example between 0.5 and 10%, most preferably in an amount between 1.25 and 5%.

The oil included in the composition is chosen to promote the retention of the antigen in oil-in-water emulsion, i.e., to provide a vehicle for the desired antigen, and preferably has a melting temperature of less than 65° C. such that emulsion is formed either at room temperature (about 20° C. to 25° C.), or once the temperature of the emulsion is brought down to room temperature. Examples of such oils include squalene, Squalane, EICOSANE™, tetratetracontane, glycerol, and peanut oil or other vegetable oils. In one specific, non-limiting example, the oil is provided in an amount between 1 and 10%, most preferably between 2.5 and 5%. The oil should be both biodegradable and biocompatible so that the body can break down the oil over time, and so that no adverse affects, such as granulomas, are evident upon use of the oil.

An adjuvant can be included in the composition. In one embodiment, the adjuvant is a mixture of stabilizing detergents, micelle-forming agent, and oil available under the name Provax® (IDEC Pharmaceuticals, San Diego, Calif.).

In another embodiment, a pharmaceutical composition includes a nucleic acid encoding a 68h05 polypeptide or immunogenic fragment thereof. A therapeutically effective amount of the 68h05 polynucleotide can be administered to a subject in order to generate an immune response. In one specific, non-limiting example, a therapeutically effective amount of the 68h05 polynucleotide is administered to a subject to treat breast or prostate cancer.

One approach to administration of nucleic acids is direct immunization with plasmid DNA, such as with a mammalian expression plasmid. As described above, the nucleotide sequence encoding 68h05, or an immunogenic peptide thereof, can be placed under the control of a promoter to increase expression of the molecule.

Immunization by nucleic acid constructs is well known in the art and taught, for example, in U.S. Pat. No. 5,643,578 (which describes methods of immunizing vertebrates by introducing DNA encoding a desired antigen to elicit a cell-mediated or a humoral response), and in U.S. Pat. No. 5,593,972 and U.S. Pat. No. 5,817,637 (which describe operably linking a nucleic acid sequence encoding an antigen to regulatory sequences enabling expression). U.S. Pat. No. 5,880,103 describes several methods of delivery of nucleic acids encoding immunogenic peptides or other antigens to an organism. The methods include liposomal delivery of the nucleic acids (or of the synthetic peptides themselves), and immune-stimulating constructs, or ISCOMS™, negatively charged cage-like structures of 30-40 nm in size formed spontaneously on mixing cholesterol and Quil A™ (saponin). Protective immunity has been generated in a variety of experimental models of infection, including toxoplasmosis and Epstein-Barr virus-induced tumors, using ISCOMS™ as the delivery vehicle for antigens (Mowat and Donachie, Immunol. Today 12:383, 1991). Doses of antigen as low as 1 jig encapsulated in ISCOMS™ have been found to produce Class I mediated CTL responses (Takahashi et al., Nature 344:873, 1990).

In another approach to using nucleic acids for immunization, a 68h05 polypeptide or an immunogenic peptide thereof can also be expressed by attenuated viral hosts or vectors or bacterial vectors. Recombinant vaccinia virus, adeno-associated virus (AAV), herpes virus, retrovirus, or other viral vectors can be used to express the peptide or protein, thereby eliciting a CTL response. For example, vaccinia vectors and methods useful in immunization protocols are described in U.S. Pat. No. 4,722,848. BCG (Bacillus Calmette Guerin) provides another vector for expression of the peptides (see Stover, Nature 351:456-460, 1991).

In one embodiment, a nucleic acid encoding a 68h05 polypeptide or an immunogenic fragment thereof is introduced directly into cells. For example, the nucleic acid may be loaded onto gold microspheres by standard methods and introduced into the skin by a device such as Bio-Rad's Helios™ Gene Gun. The nucleic acids can be “naked,” consisting of plasmids under control of a strong promoter. Typically, the DNA is injected into muscle, although it can also be injected directly into other sites, including tissues in proximity to metastases. Dosages for injection are usually around 0.5 μg/kg to about 50 mg/kg, and typically are about 0.005 mg/kg to about 5 mg/kg (see, e.g., U.S. Pat. No. 5,589,466).

In a further embodiment, a therapeutically effective amount of an antibody that specifically binds a 68h05 polypeptide can be administered to a subject, such as a subject with prostate or breast cancer. The antibody can be a humanized monoclonal antibody, or a human monoclonal antibody. Cell growth inhibiting chimeric molecules including an antibody that specifically binds 68h05 linked to a toxin (such as, but not limited to, PE linked to an anti-68h05 antibody, or a radionucleotide linked to an anti-68h05 antibody), can be prepared in pharmaceutical compositions. These cell growth inhibiting molecules can be administered to a subject by any method known to one of skill in the art. For example, to treat breast cancer, the pharmaceutical compositions of this disclosure can be administered directly into the breast tissue. To treat prostate cancer, the pharmaceutical compositions of this disclosure can be administered directly into the prostate. Metastases of breast cancer or prostate cancer may be treated by intravenous administration or by localized delivery to the tissue surrounding the tumor.

The compositions for administration will commonly comprise a solution of the cell growth inhibiting chimeric molecules dissolved in a pharmaceutically acceptable carrier, preferably an aqueous carrier. A variety of aqueous carriers can be used, e.g., buffered saline and the like. These solutions are sterile and generally free of undesirable matter. These compositions can be sterilized by conventional, well-known sterilization techniques. The compositions can contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of cell growth inhibiting molecules in these formulations can vary, and will be selected primarily based on fluid volumes, viscosities, body weight and the like in accordance with the particular mode of administration selected and the patient's needs.

In one specific, non-limiting example, a pharmaceutical composition for intravenous administration, such as an immunotoxin, would be about 0.1 to 10 mg per patient per day. Dosages from 0.1 up to about 100 mg per patient per day can be used, particularly if the agent is administered to a secluded site and not into the circulatory or lymph system, such as into a body cavity or into a lumen of an organ. Actual methods for preparing administrable compositions will be known or apparent to those skilled in the art and are described in more detail in such publications as Remingtons Pharmaceutical Sciences, 19^{th} Ed., Mack Publishing Company, Easton, Pa. (1995).

The compositions can be administered for therapeutic treatments. In therapeutic applications, a therapeutically effective amount of the composition is administered to a subject suffering from a disease, such as breast or prostate cancer. Single or multiple administrations of the compositions are administered depending on the dosage and frequency as required and tolerated by the subject. In one embodiment, the dosage is administered once as a bolus, but in another embodiment can be applied periodically until either a therapeutic result is achieved. Generally, the dose is sufficient to treat or ameliorate symptoms or signs of disease without producing unacceptable toxicity to the subject. Systemic or local administration can be utilized.

Controlled release parenteral formulations of cell growth inhibiting chimeric molecules can be made as implants, oily injections, or as particulate systems. For a broad overview of protein delivery systems (see Banja, Therapeutic Peptides and Proteins: Formulation, Processing, and Delivery Systems, Technomic Publishing Company, Inc., Lancaster, Pa., 1995). Particulate systems include microspheres, microparticles, microcapsules, nanocapsules, nanospheres, and nanoparticles. Microcapsules contain the therapeutic protein as a central core. In microspheres the therapeutic is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly (see Kreuter, Colloidal Drug Delivery Systems, J. Kreuter, ed., Marcel Dekker, Inc., New York, N.Y., pp. 219-342, 1994; Tice & Tabibi, Treatise on Controlled Drug Delivery, A. Kydonieus, ed., Marcel Dekker, Inc. New York, N.Y., pp. 315-339, 1992).

Polymers can be used for ion-controlled release. Various degradable and nondegradable polymeric matrices for use in controlled drug delivery are known in the art (Langer, Accounts Chem. Res. 26:537, 1993). For example, the block copolymer, polaxamer 407 exists as a viscous yet mobile liquid at low temperatures but forms a semisolid gel at body temperature. It has shown to be an effective vehicle for formulation and sustained delivery of recombinant interleukin-2 and urease (Johnston et al., Pharm. Res. 9:425, 1992; and Pec, J. Parent. Sci. Tech. 44(2):58, 1990). Alternatively, hydroxyapatite has been used as a microcarrier for controlled release of proteins (Ijntema et al., Int. J. Pharm. 112:215, 1994). In yet another aspect, liposomes are used for controlled release as well as drug targeting of the lipid-capsulated drug (Betageri et al., Liposome Drug Delivery Systems, Technomic Publishing Co., Inc., Lancaster, Pa., 1993). Numerous additional systems for controlled delivery of therapeutic proteins are known (e.g., U.S. Pat. No. 5,055,303; U.S. Pat. No. 5,188,837; U.S. Pat. No. 4,235,871; U.S. Pat. No. 4,501,728; U.S. Pat. No. 4,837,028; U.S. Pat. No. 4,957,735; and U.S. Pat. No. 5,019,369; U.S. Pat. No. 5,055,303; U.S. Pat. No. 5,514,670; U.S. Pat. No. 5,413,797; U.S. Pat. No. 5,268,164; U.S. Pat. No. 5,004,697; U.S. Pat. No. 4,902,505; U.S. Pat. No. 5,506,206, U.S. Pat. No. 5,271,961; U.S. Pat. No. 5,254,342 and U.S. Pat. No. 5,534,496).

In another method, antigen presenting cells (APCs) are pulsed or co-incubated with peptides comprising an epitope from 68h05 in vitro. These cells are used to sensitize CD8 cells, such as tumor infiltrating lymphocytes from breast or prostate cancer tumors or peripheral blood lymphocytes. The TILs or PBLs preferably are from the subject. However, they should at least be MHC Class-I restricted to the HLA types the subject possesses. An effective amount of the sensitized cells are then administered to the subject.

PBMCs may be used as the responder cell source of CTL precursors. The appropriate antigen-presenting cells are incubated with peptide, after which the peptide-loaded antigen-presenting cells are then incubated with the responder cell population under optimized culture conditions. Positive CTL activation can be determined by assaying the culture for the presence of CTLs that kill radio-labeled target cells, both specific peptide-pulsed targets as well as target cells expressing endogenously processed forms of the antigen from which the peptide sequence was derived.

The cells can be administered to inhibit the growth of cells of 68h05-expressing tumors. In these applications, a therapeutically effective amount of activated antigen presenting cells, or activated lymphocytes, are administered to a subject suffering from a disease, in an amount sufficient to raise an immune response to 68h05-expressing cells. The resulting immune response is sufficient to slow the proliferation of such cells or to inhibit their growth.

In a supplemental method, any of these immunotherapies is augmented by administering a cytokine, such as interleukin (IL)-2, IL-3, IL-6, IL-10, IL-12, IL-15, GM-CSF, interferons.

### Diagnostic Methods and Kits

A method is provided herein for the detection of 68h05-expressing breast or prostate cancer cells in a biological sample. The sample can be any sample that includes 68h05 polypeptide or a nucleic acid encoding 68h05 polypeptide. Such samples include, but are not limited to, tissue from biopsies, autopsies, and pathology specimens. Biological samples also include sections of tissues, such as frozen sections taken for histological purposes. Biological samples further include body fluids, such as blood, sputum, serum, or urine. A biological sample is typically obtained from a mammal, such as a rat, mouse, cow, dog, guinea pig, rabbit, or primate. In one embodiment, the primate is macaque, chimpanzee, or a human. In a further embodiment, the subject is a human with breast or prostate cancer, or is suspected of having breast or prostate cancer. In a specific, non-limiting example, the subject has metatastic breast or prostate cancer. Methods of detection include, for example, radioimmunoassay, sandwich immunoassays (including ELISA), immunofluorescence assays, Western blot, affinity chromatography (affinity ligand bound to a solid phase), and in situ detection with labeled antibodies.

In one embodiment, the detection of 68h05-expressing cells is used in staging a cancer. For example, expression of 68h05 can be used as a marker in the classification of a breast cancer, such as classifying a ductal carcinoma as stage I, IIA, IIB, IIIA, IIIB, or IV. Thus, a breast biopsy is obtained, and the expression of 68h05 is used to classify the cancer. The expression of 68h05 can be used alone, or can be used with other methods to classify the breast cancer known in the art.

In one embodiment, a method is provided for detecting a 68h05 polypeptide in a biological sample. Kits for detecting a 68h05 polypeptide of fragment thereof will typically comprise an antibody that specifically binds 68h05. In some embodiments, an antibody fragment, such as an Fv fragment is included in the kit. For in vivo uses, the antibody is preferably an scFv fragment. In a further embodiment, the antibody is labeled (e.g. fluorescent, radioactive, or an enzymatic label). The antibody can be a monoclonal or a polyclonal antibody.

In one embodiment, a kit includes instructional materials disclosing means of use of an antibody that specifically binds a 68h05 polypeptide or fragment thereof (e.g. for detection of 68h05 expressing breast or prostate cancer cells in a sample). The instructional materials may be written, in an electronic form (e.g. computer diskette or compact disk) or may be visual (e.g. video files). The kits may also include additional components to facilitate the particular application for which the kit is designed. Thus, for example, the kit may additionally contain means of detecting a label (e.g. enzyme substrates for enzymatic labels, filter sets to detect fluorescent labels, appropriate secondary labels such as a secondary antibody, or the like). The kits may additionally include buffers and other reagents routinely used for the practice of a particular method. Such kits and appropriate contents are well known to those of skill in the art.

In one embodiment of the present invention, the diagnostic kit comprises an immunoassay. Although the details of the immunoassays may vary with the particular format employed, the method of detecting a 68h05 polypeptide or fragment thereof in a biological sample generally comprises the steps of contacting the biological sample with an antibody which specifically reacts, under immunologically reactive conditions, to 68h05. The antibody is allowed to specifically bind under immunologically reactive conditions to form an immune complex, and the presence of the immune complex (bound antibody) is detected directly or indirectly.

In an alternative set of embodiments, kits can be provided for detecting nucleic acids encoding 68h05 or a fragment thereof in a biological sample. For example, samples from a subject can be tested to determine whether nucleic acids encoding 68h05 protein are present. In one embodiment, an amplification procedure is utilized to detect nucleic acids encoding 68h05. In another embodiment, a blotting procedure (e.g. Northern Blot or Dot Blot) is used to detect the presence of nucleic acids encoding 68h05. Thus, a kit can include probes or primers that specifically hybridize to nucleic acids encoding 68h05.

In one embodiment, a kit provides at least one primer that can be used to amplify nucleic acid encoding 68h05 from a biological sample. Conveniently, the amplification is performed by polymerase chain reaction (PCR). A number of other techniques are, however, known in the art and are contemplated for use (for example, see Marshall, U.S. Pat. No. 5,686,272, discloses the amplification of RNA sequences using ligase chain reaction, or “LCR,” Landegren et al., Science 241:1077, 1988; Wu et al., Genomics, 4:569, 1989; Barany, in PCR Methods and Applications 1:5, 1991; and Barany, Proc. Natl. Acad. Sci. USA 88:189, 1991). In one specific, non-limiting example, RNA can be reverse transcribed into DNA and then amplified by LCR, PCR, or other methods. An exemplary protocol for conducting reverse transcription of RNA is taught in U.S. Pat. No. 5,705,365. Selection of appropriate primers and PCR protocols are taught, for example, in Innis et al., eds., PCR Protocols 1990 (Academic Press, San Diego, Calif.).

In one embodiment, the kit includes instructional materials disclosing the manner of use for the primer or probe. The kits may also include additional components to facilitate the particular application for which the kit is designed. The kits may additionally include buffers and other reagents routinely used for the practice of a particular method. Such kits and appropriate contents are well known to those of skill in the art.

The disclosure is illustrated by the following non-limiting Examples.

### EXAMPLES

### Example 1

### Identification of 68h05

To discover new antigens as targets for immunotherapies of breast cancers and secreted proteins for use as diagnostic markers, a molecular approach was taken to identify membrane and secreted proteins that are present in breast cancers but are not expressed in normal essential tissues (see Egland et al., Proc. Natl. Acad. Sci 100(3): 1099, 2003). The procedure is summarized as follows: secretory and integral membrane proteins were translated from mRNA on membrane-bound ribosomes associated with the endoplasmic reticulum. Isolation of the membrane-associated polyribosomal RNA produced an enriched population of transcripts that encode membrane and secretory proteins. A high-quality cDNA library was generated that is enriched with genes that encode membrane and secreted proteins using membrane-associated RNA from six cell lines: four different breast cancer cell lines, one normal breast cell line, and a prostate cancer cell line. This cDNA library was subtracted with RNA from a pool of five libraries derived from liver, kidney, brain, lung, and muscle to enrich for differentially expressed genes in breast and prostate cancer while removing or reducing ubiquitously expressed genes. Briefly, the methods include:

Primers: The following primers were used.

| | |
|---|---|
| 68_h05-C-For | |
| (5′-CTCTAGACGCGCTGCACCTGC-3′), | SEQ ID NO: 8 |
| and | |
| R (reverse) 68_h05-C-Rev | |
| (5′-CCGCTGCCGCCCAAGCCTG-3′), | SEQ ID NO: 9) |
| Actin-For | |
| (5′-GCATGGGTCAGAAGGAT-3′), | SEQ ID NO: 10 |
| and | |
| Actin-Rev | |
| (5′-CCAATGGTGATGACCTG-3′), | SEQ ID NO: 11 |

Cell Culture: MCF7, SK-BR-3, ZR-75-1, MDA-MB-231, and LNCaP cell lines were maintained as recommended by the American Type Culture Collection. The hTERT-HME1 cell line (CLONTECH) was maintained according to the manufacturer's instructions.

Isolation of Membrane-Associated Polyribosomal RNA: MCF7, SK-BR-3, ZR-75-1, MDA-MB-231, hTERT-HME1, and LNCaP cells (1×10⁸ cells per prep) were individually treated with 50 μM cycloheximide (Sigma) for 10 minutes at 37° C. The cells were washed twice with ice-cold PBS solution and scraped from the dish into a 50-ml conical tube. The cells were centrifuged and resuspended to 1.25×10⁸ cells per ml in hypotonic buffer [10 mM KCl/1.5 mM MgCl₂/10 mM Tris-HCl, pH 7.4/200 units/ml RNase inhibitor (Roche, Indianapolis)]. The cells were placed on ice to swell for 10 minutes and ruptured with a Dounce by using pestle B (Kontes). The membrane-associated polyribosomes and cytosolic polyribosomes were separated by isopycnic centrifugation in a discontinuous sucrose density gradient at 90,000×g for 15 hours at 4° C. (Mechler, Methods Enzymol. 152, 241-248, 1987). The total RNA was isolated from the membrane polyribosomal fraction by using Trizol LS reagent (Invitrogen Life Technologies, Carlsbad, Calif.). The quality of the total RNA was verified by using the Agilent 2100 bioanalyzer. Individual preps of membrane-associated polyribosomal RNA from each cell line were pooled as follows: 300 μg each isolated from MCF7, SK-BR-3, ZR-75-1, MDA-MB-231, and hTERT-HME1 cells and 200 μg from LNCaP cells. Of the pooled RNA, 100 μg was saved for future analysis, and 1.6 mg was given to Invitrogen Life Technologies for library construction.

Generation of the MAPcL: mRNA was isolated from the pooled total membrane-associated polyribosomal RNA, and cDNA was generated by using an oligo(dT) primer by Invitrogen Life Technologies. The cDNA fragments were cloned directionally into the EcoRV and NotI sites of pCMVSport6.0 (Invitrogen Life Technologies), resulting in the destruction of the EcoRV site. The library was electroporated into Escherichia coli EMDH10B cells, and the titer of the library was determined. Twenty-three clones were picked randomly to determine the average insert size of the library.

Subtraction of the MAPcL: Clones (5×10⁶) of the MAPcL were amplified 26,000-fold by Invitrogen Life Technologies by using their semisolid agarose procedure, which minimizes clone bias that normally occurs during liquid amplification. A driver library was created by pooling Invitrogen Life Technologies' premade liver, brain, kidney, lung, and skeletal muscle libraries in equimolar amounts. The amplified MAPcL was subtracted with the driver library (Li et al., BioTechniques 16:722-729, 1994). The subtracted library contains 1.3×10⁷ colony-forming units total with an average insert size of 1,800 bp.

Sequencing of the Subtracted and Unsubtracted MAPcL: The 5′ sequencing reactions were performed at the Advanced Technology Center (Gaithersburg, Md.) by using the M13 reverse primer.

Sequence Identification: Sequences of clones from the MAPcL were identified initially by comparison to the NCBI RefSeq, GenBank, and expressed sequence tags databases (dbEST) using BLAST (Pruitt et al., J. Mol. Biol. 215: 403-410, 1990). Full-length clones were identified as MAPcL sequences with a hit to a RefSeq protein at 70% identity or better and an alignment starting at amino acid 1 of the RefSeq protein. Membrane and secreted proteins were identified by using Gene Ontology Consortium (GO) classifications associated with RefSeq genes. The NIH_MGC_{—}87 cDNA library from the NIH Mammalian Gene Collection was used as a control for membrane and secreted proteins (Strausberg et al., Science 286: 455-457, 1999). This library contains >19,000 ESTs and was made from an adenocarcinoma breast tissue-derived cell line. MAPcL sequences representing unknown genes were classified by tissue expression by using EST sequences from the dbEST.

Web Sites: UniGene, RefSeq, dbEST, and GenBank sequence databases, the BLAST program, LocusLink, OMIM, and the CGAP project can be accessed from the NCBI website available on the internet. The GO database can be obtained from the gerontology website. The NIH_MGC_{—}87 cDNA library can be obtained from the MGC website maintained by the National Institutes of health. The GoldenPath genome build and annotation databases can be accessed from the University of Southern California website. For specific website addresses see Egland et al., Proc. Natl. Acad. Sci 100(3):1099, 2003. All database versions except GoldenPath were taken as a snapshot from public releases available as of Mar. 14, 2002. Genome sequences and annotations were taken from the December 2001 build of GoldenPath.

To generate a breast and prostate cancer cDNA library enriched with genes that encode membrane and secreted proteins, membrane-associated polyribosomal RNA was isolated from four breast cancer cell lines (MCF7, ZR-75-1, SK-BR-3, and MDA-MB-231), one telomerase immortalized normal breast cell line (hTERT-HME1), and the prostate cancer cell line (LNCaP) that produces prostate-specific antigen. The addition of the prostate cancer cell line RNA served two purposes. It served as a test of this approach because if our hypothesis was correct, the library was expected to be enriched in prostate-specific antigen. Also, it allows the discovery of unknown genes expressed in prostate cancers.

It was shown previously by using cDNA microarray analysis that there are two main subgroups of breast tumors based on their gene-expression profiles: estrogen receptor (ER)-positive and ER-negative (Perou et al., Nature 406:747-752, 2000). In addition, the overexpression of ErbB2 correlated with low levels of the ER. Because there are numerous breast cancer cell lines available, four were chosen to represent the recognized range of phenotypic diversity of breast tumors. MCF7 and ZR-75-1 both express the ER and express ErbB2 at low levels. SK-BR-3 and MDA-MB-231 do not express the ER. SK-BR-3 contains gene amplifications of ErbB2, whereas MDA-MB-231 expresses ErbB2 at low levels. Membrane-associated polyribosomal RNA was isolated individually from the six cell lines, and the RNA was pooled. A cDNA library was generated from the pooled membrane-associated polyribosomal RNA as described above. The library contains 2.01×10⁷ colony-forming units total with an average insert size of 2 kb.

To remove ubiquitously expressed genes and enrich for genes specifically expressed in breast and prostate cancers, the initial MAPcL was subtracted by using biotinylated RNA generated from five normal libraries: brain, liver, lung, kidney, and skeletal muscle as described above. The efficiency of subtraction was determined by measuring the level of a housekeeping gene, eukaryotic elongation factor 1, or EEF1A1. The EEF1Al gene was reduced by 85-fold in the subtracted library.

To determine which genes are represented in the initial and subtracted MAPcLs, one sequencing reaction was performed on the 5′ end of 943 unsubtracted clones and 15,581 subtracted clones that were chosen randomly. The sequences from the two libraries were compared with known genes in RefSeq, a public database of curated genes (Pruitt & Maglott, Nucleic Acids Res. 29:137-140, 2001). The most abundant known genes in the initial and subtracted libraries are listed in Egland et al., Proc. Natl. Acad. Sci. 100(3):1099, 2003, which is incorporated by reference in its entirety. Although the initial library was made from enriched membrane-associated polyribosomal RNA, it still contained cDNAs derived from highly expressed genes that encode soluble, housekeeping proteins such as glyceraldehyde-3-phosphate dehydrogenase and EEF1A1. Subtraction of the initial library successfully removed these contaminating sequences such that of the 15,581 sequenced clones, glyceraldehyde-3-phosphate dehydrogenase was not present and only one clone encoded EEF1A1. Furthermore, the most abundant gene was prostate-specific antigen, a secreted protein highly expressed in the LNCaP cell line.

Because all sequencing reactions were performed from the 5′ end, it is possible to determine what percentage of the cDNA inserts encode full-length transcripts of known genes. Using BLASTX to compare translated MAPcL sequences to the RefSeq protein database, it was determined that 30% of the MAPcL sequences contain the 5′ end of the encoded proteins (Altschul et al., J. Mol. Biol. 215:403-410, 1990).

To quantify the enrichment of clones encoding membrane and secreted proteins, MAPcL sequences representing known genes were assessed by using the GO database (The Gene Ontology Consortium, Genome Res. 11:1425-1433, 2001). According to the cellular location classification from the GO database, 49% of the known genes in the subtracted MAPcL encode membrane or secreted proteins. In contrast, only 14% of known genes encode membrane or secreted proteins from a control library derived from unfractionated mRNA from an adenocarcinoma breast tissue.

The 15,581 sequences from clones of the subtracted MAPcL were classified as either known or unknown based on a BLAST analysis (Altschul et al., J. Mol. Biol. 215:403-410, 1990). Sequences were labeled as known if they aligned to a gene sequence in the RefSeq database; otherwise, they were labeled unknown. Of 15,581 MAPcL sequences, 10,506 sequences aligned with 3,814 RefSeq genes. The remaining 5,075 unknown sequences were divided into three groups: (i) 4,074 sequences, aligned with 2,382 UniGene clusters that were not associated with known genes, (ii) 354 sequences, representing 342 unique transcripts, overlapped with ESTs that were not part of any UniGene clusters, and (iii) 647 sequences, representing 457 unique transcripts, which did not overlap any known sequences (Boguski et al., Nat. Genet. 4:332-333, 1993).

The 5,075 sequences from the subtracted MAPcL that are not associated with known genes were examined to narrow the search for genes encoding potential immunotherapy targets. Candidate sequences chosen for further study either align to EST sequences derived only from nonessential tissue libraries, have alternative splice forms different from ESTs derived from essential tissues, or do not align with any ESTs. The sequences were aligned to the human genome using BLAST from the GoldenPath project (December 2001 build, see above) and surveyed the genomic region around these sequences for evidence of gene structure based on other ESTs that were also aligned to the genome (Kent & Haussler, Genome Res. 11I:1541-1548, 2001; Kent et al., Genome Res. 12:996-1006, 2002; Kent, Genome Res. 12:656-664, 2002). MAPcL sequences that seemed to represent the 5′ end of genes containing ESTs from excluded tissues were eliminated. In addition, all candidate MAPcL sequences contain a predicted ORF based on the sequence obtained from one reaction from the 5′ end.

The 68h05 nucleic acid sequence aligned with part of UniGene cluster Hs.443112. This cluster contains a single hypothetical mRNA and 23 ESTs. This cluster was not previously associated with a known gene.

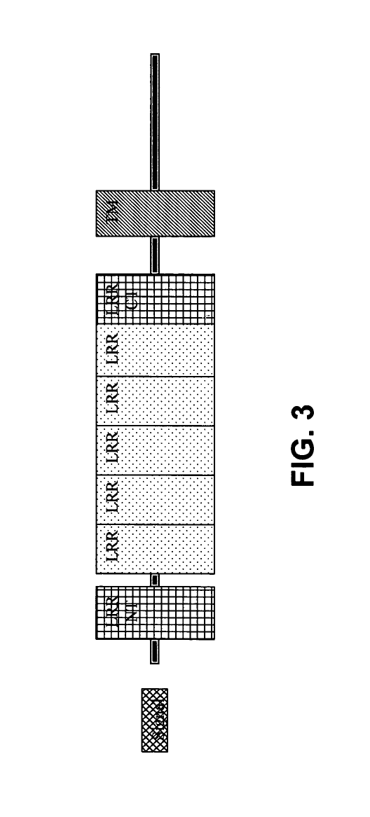

A diagram of the alignment of the 68h05 consensus sequence and MAPcL clone cDNA sequence with human chromosome 9 is shown in FIG. 1. The nucleic acid sequence of 68h05 is shown in FIG. 2A, and the amino acid sequence of 68h05 is shown in FIG. 2B. A schematic diagram of the amino acid sequence is shown in FIG. 3.

### Example 2

### Expression of 68h05 in Breast Cancer, Breast Cancer Cell Lines, and a Prostate Cancer Cell Line

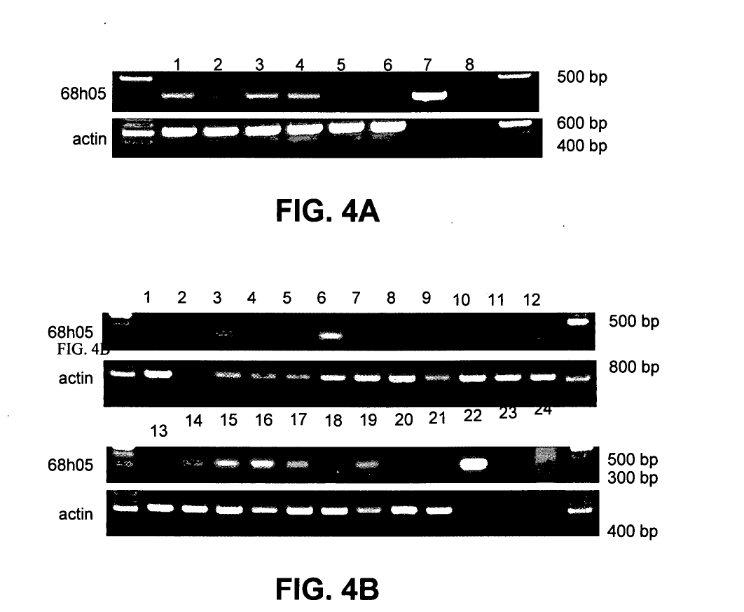

Total RNA was isolated from frozen breast tumor samples acquired from the Cooperative Human Tissue Network and tissue culture cell lines by using the StrataPrep Total RNA miniprep kit (Stratagene) according to the manufacturer's instructions. To generate single-stranded cDNAs, total RNA (5 μg) was used with the First-Strand cDNA synthesis kit by using random hexamer priming according to manufacturer instructions (Amersham Pharmacia). PCRs were performed by using the following protocol: initial denaturation at 94° C. for 3 minutes, 35 cycles of denaturation at 94° C. for 1 minutes, annealing at 60° C. for 1 minutes, elongation at 72° C. for 1 minutes, and a final 5-minute extension at 72° C. The results are shown in FIG. 4. Expression was detected in the LNCaP cell line (prostate cancer) and four breast cancer cell lines, but not in the normal breast cell line (FIG. 4A). In addition, 16 out of 21 breast tumors (FIG. 4B), and 3 of 3 prostate tumors (data not shown) expressed 68h05. Separate PCR reactions were performed using actin primers to verify the quality of the generated cDNA (FIG. 4).

### Example 3

### Expression Analysis of 68h05 in Normal Tissues

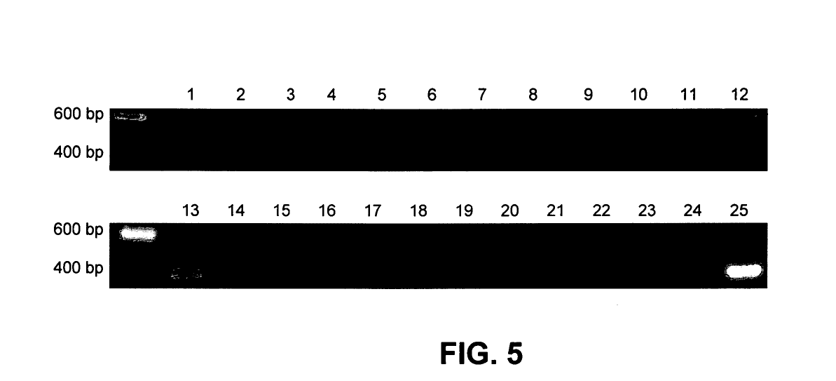

PCR analysis was performed using a Rapid Scan panel (OriGene Technologies) containing cDNA samples derived from 24 normal tissues (FIG. 5). Similar PCR conditions were used as above in example 2. The rapid scan panel revealed an abundant 430-bp PCR product from cDNA derived from salivary gland (FIG. 5, lane 13) and a weak 430-bp PCR product in prostate (FIG. 5, lane 19). To verify the quality of the cDNA templates, separate PCR reactions were performed using actin primers, and bands of equal intensity were observed.

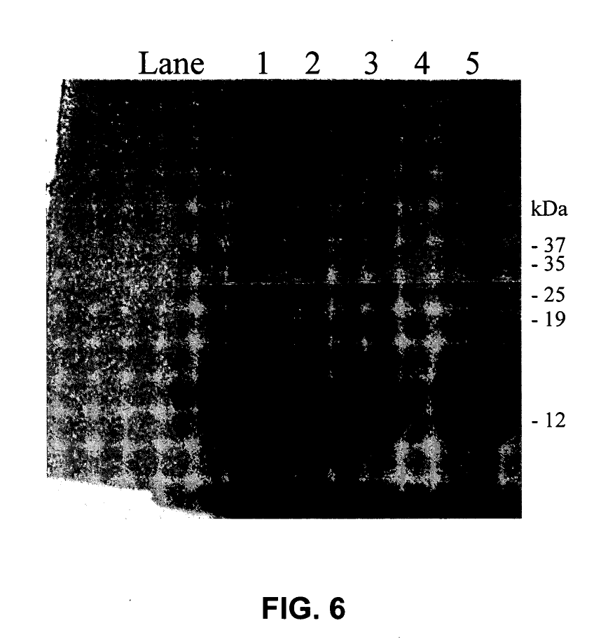

The cDNA clone 68h05 in plasmid vector pCMVSport6.0 was transcribed and translated in vitro using the TNT® Coupled Reticulocyte Lysate System (Promega cat. #L4601). 1.0 μg of the plasmid DNA was used in the system as described on page 6 of the Promega manual #TB 126. ³⁵S labeled cysteine was used instead of methionine. A negative control was done by replacing plasmid DNA with water. A positive control was done by using a plasmid encoding BASE, a 19 kDa protein (Egland et al.). The ³⁵S labeled products were separated by PAGE. The results are shown FIG. 6. A 35 kDa band was observed corresponding to the 68h05 protein.

### Example 4

### Antibodies to 68h05 and Radioimmunoassay to Detect 68h05

To generate antibodies against 68h05, rabbits were injected with a 68h05 peptide, CRARRRRLRTAALRPPRPPDPNPDPDPHG (SEQ ID NO: 12). This rabbit antiserum is able to detect 68h05 when 293T cells are transfected with a 68h05-Myc/His expression plasmid. The 68h05-Myc/His specific band has a molecular weight of 37 kDa. It should be noted that the 68h05 expression plasmid has an expression pattern similar to that of the transferrin receptor. 68h05 was located in the same compartment as transferrin receptor by fluorescence localization experiments.

The following example sets forth an exemplary protocol for a radioimmunoassay to detect the presence of 68h05 in a sample.

Radiolabeling of 68h05

Two and a half micrograms of chemically synthesized or E. coli expressed 68h05 are labeled with ¹²⁵I using the chloramine T method (Hunter and Greenwood, Nature 194:495, 1962). The labeled protein is then purified using a PD-10 column (Amersham Pharmacia Biotech).

Anti-68h05 Antibody

Anti-68h05 antibodies are prepared by using proteinA purified antisera from rabbits immunized with a Pseudomonas exotoxin (PE) -68h05 fusion protein using standard techniques (Bruggeman et al., BioTechniques 10:202, 1992). Alternatively, antibodies are prepared using the method described above.

Standard Curve

A standard curve is established by mixing a fixed amount of labeled 68h05 (˜0.2 ng at about 170 μCi/μg) with different concentrations of unlabeled 68h05 (0.1 ng-50 ng) in 250 μl buffer (PBS with 0.25% bovine serum albumin) containing 1 μg of anti-68h05 antibody. The samples are incubated at room temperature for 4 hours. ProteinA sepharose beads are added and incubated for another hour. Finally the beads are collected by centrifugation and washed with buffer 3 times. The remaining bead pellet is measured for radioactivity in a gamma counter.

Sample Measurement

To measure the amount of 68h05 in a tissue extract or a protein extract from a cell culture the same procedure is used, but with the sample substituted for the known amounts of the protein used in the standard curve description.

### Example 5

### Kit for the Detection of Metastatic Breast or Prostate Cancer

Breast cancer and prostate cancer are known to metastasize to other areas of the body, such as bone. Antibodies to a 68h05 polypeptide can be used to detect breast or prostate cancer cells at locations other than the breast.

In order to determine if a metastatic tumor originates in the breast or prostate, the expression of 68h05 is assessed. Specifically, a kit is utilized that provides an immunoassay that can be used to confirm that the cancer cells are of breast or prostate origin.

A biological sample of the metastasis is obtained. In one example, the sample is a bone marrow sample. Non-specific immunoreactive sites on biological sample are blocked with a commercially available blocking agent, such as 10% bovine serum albumin in phosphate buffered saline (PBS), for thirty minutes at room temperature. The sample is then contacted with a mouse monoclonal antibody that specifically binds 68h05 for an incubation period sufficient to allow formation of an immune complex (e.g. ten minutes to three hours at room temperature in a solution of 1% BSA). The presence of the immune complex (bound antibody) is detected by incubating the sample with a commercially available labeled secondary antibody that specifically binds the 68h05 antibody. For example, a fluorescent labeled (e.g. fluorescein isothiocyanate, FITC) goat anti-mouse antibody is diluted 1:100 in PBS/1% BSA and incubated with the sample for an amount of time sufficient to form an immune complex (e.g. ten minutes to about two hours at room temperature). The samples are then processed to determine binding of the second antibody (e.g. detection of fluorescence). A positive signal indicates that the metastasis is of breast or prostate origin.

### Example 6

### Activation of T Cells Using 68h05

Methods for evaluating immunogenicity of peptides are known. Immunogenicity be evaluated by, for example, evaluation of primary T cell cultures from normal individuals (see, e.g., Wentworth et al., Mol. Immunol. 32:603, 1995; Celis et al., Proc. Natl. Acad. Sci. USA 91:2105, 1994; Tsai et al., J. Immunol. 158:1796, 1997; Kawashima et al., Human Immunol. 59:1, 1998); by immunization of HLA transgenic mice (see, e.g., Wentworth et al., J. Immunol. 26:97, 1996; Wentworth et al., Int. Immunol. 8:651, 1996; Alexander, et al., J. Immunol. 159:4753, 1997), and by demonstration of recall T cell responses from patients who have been effectively vaccinated or who have a tumor (see, e.g., Rehermann et al., J. Exp. Med 181:1047, 1995; Doolan et al., Immunity 7:97, 1997; Bertoni et al., J. Clin. Invest. 100:503, 1997; Threlkeld et al., J. Immunol. 159:1648 1997; Diepolder et al., J. Virol. 71:6011, 1997).

In choosing CTL-inducing peptides of interest, peptides with higher binding affinity for Class I HLA molecules can be utilized. Peptide binding is assessed by testing the ability of a candidate peptide to bind to a purified HLA molecule in vitro.

Based on the polypeptide sequence of 68h05, amino acid sequences bearing motifs for any particular HLA molecule can be identified. Peptides including these motifs can be prepared by any of the typical methods (e.g., recombinantly, chemically, etc.). Because 68h05 is a self protein, the amino acid sequences bearing HLA binding motifs are those that encode subdominant or cryptic epitopes. Those epitopes are identified by a lower comparative binding affinity for the HLA molecule with respect to other epitopes in the molecule or compared with other molecules that bind to the HLA molecule.

Polypeptides that include an amino acid sequence from 68h05 that, in turn, include an HLA binding motif also are useful for eliciting an immune response. This is because, in part, such proteins will be processed by the cell into a peptide that can bind to the HLA molecule and that have a 68h05 epitope.

A complex of an HLA molecule and a peptidic antigen acts as the ligand recognized by HLA-restricted T cells (Buus et al., Cell 47:1071, 1986; Babbitt et al., Nature 317:359, 1985; Townsend and Bodmer, Annu. Rev. Immunol. 7:601, 1989; Germain, Annu. Rev. Immunol. 11:403, 1993). Through the study of single amino acid substituted antigen analogs and the sequencing of endogenously bound, naturally processed peptides, critical residues that correspond to motifs required for specific binding to HLA antigen molecules have been identified (see, e.g., Southwood et al., J. Immunol. 160:3363, 1998; Rammensee et al., Immunogenetics 41:178, 1995; Rammensee et al., J. Curr. Opin. Immunol. 10:478, 1998; Engelhard, Curr. Opin. Immunol. 6:13, 1994; Sette and Grey, Curr. Opin. Immunol. 4:79, 1992).

Furthermore, x-ray crystallographic analysis of HLA-peptide complexes has revealed pockets within the peptide binding cleft of HLA molecules which accommodate, in an allele-specific mode, residues borne by peptide ligands; these residues in turn determine the HLA binding capacity of the peptides in which they are present (e.g., Madden, Annu. Rev. Immunol. 13:587, 1995; Smith, et al., Immunity 4:203, 1996; Fremont et al., Immunity 8:305, 1998; Stern et al., Structure 2:245, 1994; Jones, Curr. Opin. Immunol. 9:75, 1997; Brown et al., Nature 364:33, 1993).

Accordingly, the definition of Class I and Class II allele-specific HLA binding motifs, or Class I or Class II supermotifs allows identification of regions within 68h05 that have the potential of binding particular HLA molecules.

One method of identifying genes encoding antigenic determinants is as follows: Tumor infiltrating lymphocytes (TILs) from a subject with breast or prostate cancer are grown and tested for the ability to recognize the autologous cancer in vitro. These TILs are administered to the subject to identify the ones that result in tumor regression. The TILs are used to screen expression libraries for genes that express epitopes recognized by the TILs. Subjects then are immunized with these genes. Alternatively, lymphocytes are sensitized in vitro against antigens encoded by these genes. Then the sensitized lymphocytes are adoptively transferred into subjects and tested for their ability to cause tumor regression. Rosenberg et al., Immunol. Today 18:175, 1997.

To ensure that 68h05 elicits a CTL response to 68h05 in vivo (or, in the case of Class II epitopes, elicits helper T cells that cross-react with the wild type peptides), the 68h05 can be used to immunize T cells in vitro from individuals of the appropriate HLA allele. Thereafter, the immunized cells' capacity to induce lysis of 68h05-sensitized target cells is evaluated.

More generally, 68h05 peptides can be synthesized and tested for their ability to bind to HLA proteins and to activate HTL or CTL responses, or both.

Conventional assays utilized to detect T cell responses include proliferation assays, lymphokine secretion assays, direct cytotoxicity assays, and limiting dilution assays. For example, antigen-presenting cells that have been incubated with a peptide can be assayed for the ability to induce CTL responses in responder cell populations.

PBMCs can be used as the responder cell source of CTL precursors. The appropriate antigen-presenting cells are incubated with peptide, after which the peptide-loaded antigen-presenting cells are then incubated with the responder cell population under optimized culture conditions. Positive CTL activation can be determined by assaying the culture for the presence of CTLs that kill radiolabeled target cells, both specific peptide-pulsed targets as well as target cells expressing endogenously processed forms of the antigen from which the peptide sequence was derived.

A method which allows direct quantification of antigen-specific T cells is staining with fluorescein-labeled HLA tetrameric complexes (Altman et al., Proc. Nail. Acad. Sci. USA 90:10330, 1993; Altman et al., Science 274:94, 1996). Alternatively, staining for intracellular lymphokines, interferon-γ release assays or ELISPOT assays, can be used to evaluate T cell responses.

CTL activation may be assessed using such techniques known to those in the art such as T cell proliferation and secretion of lymphokines, e.g. IL-2 (see e.g. Alexander et al., Immunity 1:751-761, 1994).

In one specific, non-limiting example, transgenic mice that express a chimeric human Class I major histocompatibility (MHC) Class I molecule composed of the α1 and α2 domains of HLA-A2.1 and the α3, transmembrane and cytoplamic domains of H2-kb HLA transgenic mice (see, e.g., Wentworth et al., J. Immunol. 26:97, 1996; Wentworth et al., Int. Immunol. 8:651, 1996; Alexander et al., J. Immunol. 159:4753, 1997) are immunized with plasmid DNA encoding 68h05. Specifically, each mouse is injected intramuscularly with 100 μg of plasmid five times every three weeks. After the final immunization CD8+ cells are partially purified using antibody-coated magnetic beads and re-stimulated with syngeneic spleenocytes for one week in T-stim media. Specifically, cells from the immunized transgenic mice are co-cultured either with stimulating spleenocytes (e.g., 3.5×10⁶ spleenocytes) pulsed with various concentrations (100, 0.1 or 0.0001 μM) 68h05 peptide or in the presence of free peptide (1 μM) in a 24 well plate containing 2 ml of a 1:1 mixture of RPMI1640 media and Eagle-Hanks amino acid medium supplemented with L-glutamine, sodium pyruvate, non-essential amino acids, antibiotics (penicillin, streptomycin, 5×10⁻⁵ M 2-mercaptoetanol, 10% fetal calf serum, and 10% T-stim (Collaborative Biomedical Products, Bedford, Mass.). On day seven, a cytoxic T lymphocyte (CTL) assay is carried out using LnCAP cells expressing 68h05 as target cells in the presence of peptides fragments of 68h05. For example, LnCAP target cells (1×10⁶) are labeled with 300 μCi of Na₂⁵¹CrO₄ in 200-250 μl for 2 hours at 37° C. For test samples, targets are pulsed with peptide during labeling. Cells are then washed and added to wells along with the appropriate number of effector cells in 96-well round bottom plates. After four hours, supernatents are harvested and counted in an ISOMEDIC gamma counter (ICN). The mean of triplicate samples and percent of ⁵¹Cr release is calculated (see Alexander-Miller et al., Proc. Natl. Acad. Sci. USA 93:4102). The results demonstrate that 68h05 peptides can be used to induce cytotoxic activity against LnCAP cells.

### Example 7

### Production of an Immune Response Against 68h05 in a Primate

The prostate gland of the rhesus monkey is structurally and functionally similar to the human prostate (Wakui et al., J. Anat. 181:121, 1992; U.S. Pat. No. 6,165,460). Thus, juvenile male rhesus monkeys (Macaca mulatta), ages 1 to 2 years, are assigned to groups (e.g., three vaccination groups of four animals each, a low dose, a high dose and a control group). One animal from each group is surgically prostatectomized to parallel two situations with regard to potential therapy in humans: (a) prostate intact, with primary and/or metastatic disease; or (b) patients prostatectomized with prostate cancer metastatic deposits. Animals are immunized 3 times over a two month period with a recombinant virus (e.g. a pox virus, see U.S. Pat. No. 6,165,460). For example, a dose of either 1×10⁷ or 1×10⁸ PFU of a recombinant pox virus encoding 68h05 is administered to 4 animals by skin scarification. A control vector (e.g. V-Wyeth, 1×10⁸ PFU) is administered to a control group of animals.

Physical examinations are performed on ketamine (Ketamine® HCl, 10 mg/kg I.M.) sedated animals. Rectal temperatures and weights are recorded for each monkey on a weekly basis. The vaccination site is observed and erythema and swelling of the vaccination site are measured by caliper. Each animal is examined for regional lymphadenopathy, hepatomegaly, and splenomegaly. Any other gross abnormalities were also recorded.

Blood is obtained by venipuncture from the femoral vein of ketamine sedated animals before and after each immunization. A complete blood count, differential, hepatic and renal chemistry evaluation is performed on each monkey. Results are compared to normal primate values. Circulating levels of 68h05 before and after immunization are analyzed (e.g. by immunoassay or Northern blot).

Prior to each immunization and 2 weeks following each immunization, anti-68h05 antibody is quantified by ELISA. Microtiter plates are coated with purified 68h05 (e.g. 100 ng/well,), ovalbumin (100 ng/well, Sigma), or 1×10⁷ PFU/well UV-inactivated V-Wyeth in phosphate buffered saline (PBS). The plates are blocked (e.g. using 2% BSA in PBS), dried, and stored at −20° C. until used. The plates are incubated with serum (e.g. diluted 1:5), as well as a monoclonal antibody for 68h05 as a standard control, for 24 hours at 4° C. Plates are washed several times (e.g. with PBS containing 1% BSA), and incubated with a commercially labeled antibody that specifically binds the anti-68h05 monoclonal antibody. An appropriate reagent system is used to visualize antibody binding. For example the antibody is labeled with horseradish peroxidase (HRP), and detected by HRP substrate system (Kirkegaard & Perry Laboratories, Gaithersburg, Md.) according to the manufacture's instructions. The absorbance of each well is read at 405 nm using a Bio-Tek EL310 microplate ELISA reader (Winooski, Vt.).

Sera from each monkey is analyzed by ELISA for immunoreactivity to 68h05. Sera obtained from monkeys prior to vaccination are also analyzed, and are negative for reactivity to 68h05. 68h05 specific T cell responses in monkeys immunized with 68h05 containing vector or control vector are also analyzed using a lymphoproliferative assays using peripheral blood mononuclear cells.

It will be apparent that the precise details of the methods or compositions described may be varied or modified without departing from the spirit of the described invention. We claim all such modifications and variations that fall within the scope and spirit of the claims below.

1. An isolated polypeptide comprising an amino acid sequence set forth as SEQ ID NO: 1 or an immunogenic fragment thereof comprising at least eight consecutive amino acids 1-64 of SEQ ID NO: 1, amino acids 173-190 of SEQ ID NO: 1, amino acids 215-228 of SEQ ID NO: 1, 290-318 of SEQ ID NO: 1, or amino acids 261-334 of SEQ ID NO: 1.

2. The isolated polypeptide of claim 1, comprising an amino acid sequence set forth as SEQ ID NO: 1.

3. The isolated polypeptide of claim 1, comprising at least eight consecutive amino acids 1-64 of SEQ ID NO: 1, amino acids 173-190 of SEQ ID NO: 1, amino acids 215-228 of SEQ ID NO: 1, 290-318 of SEQ ID NO: 1, or amino acids 261-334 of SEQ ID NO: 1.

4. The isolated polypeptide of claim 3, comprising an amino acid sequence as set forth as acids 290-318 of SEQ ID NO: 1.

5. The isolated polypeptide of claim 1, comprising at least one amino acid sequence set forth as
(a) amino acids 1-64 of SEQ ID NO: 1;
(b) amino acids 173-190 of SEQ ID NO: 1;
(c) amino acids 215-228 of SEQ ID NO: 1; or
(d) amino acids 261-334 of SEQ ID NO: 1.

6. An isolated nucleic acid molecule encoding the polypeptide of claim 1.

7. The isolated nucleic acid molecule of claim 6, comprising a nucleic acid sequence as set forth as SEQ ID NO: 2.

8. The isolated nucleic acid sequence of claim 6, operably linked to a promoter.

9. An expression vector comprising the nucleic acid sequence of claim 6.

10. A host cell transfected with the nucleic acid sequence of claim 6.

11. The host cell of claim 10, wherein the host cell is a mammalian cell.

12. An antibody that specifically binds the polypeptide of claim 1.

13. The antibody of claim 12, wherein the antibody is a monoclonal antibody.

14. The antibody of claim 12 comprising a detectable label.

15. The antibody of claim 12, wherein the label is a fluorescent, enzymatic or radioactive label.

16. The antibody of claim 12 conjugated to a toxin.

17. A method for detecting breast or prostate cancer in a subject, comprising
contacting a sample obtained from the subject with the antibody of claim 12 for a sufficient amount of time to form an immune complex;
detecting the presence the immune complex, wherein the presence of an immune complex demonstrates the presence of breast or prostate cancer in the subject.

18. The method of claim 17, wherein the sample is a breast biopsy, prostate biopsy, blood, serum, or urine sample.

19. The method of claim 17, wherein the sample is a biopsy sample of non-breast origin.

20. The method of claim 17, wherein the antibody is labeled.

21. A method for detecting a breast or prostate cancer in a subject, comprising
detecting the expression of the polypeptide of claim 1 in a sample from the subject, wherein an increase in the expression of the polypeptide as compared to a control indicates the presence of the breast or prostate cancer.

22. The method of claim 21, wherein detecting the expression of polypeptide comprises detecting a polypeptide having a sequence set forth as SEQ ID NO: 1 in the sample.

23. The method of claim 22, wherein detecting the expression of the polypeptide comprises
contacting the sample with an antibody that specifically binds the polypeptide for a sufficient amount of time to form an immune complex; and
detecting the presence of the immune complex.

24. The method of claim 21, wherein detecting the expression of the polypeptide comprises detecting the presence of mRNA encoding the polypeptide.

25. The method of claim 24, wherein detecting the presence of mRNA encoding the polypeptide comprises a Northern Blot analysis, an RNA Dot Blot, or a reverse transcriptase polypermase chain reaction (RT-PCR) assay.

26. A method for producing an immune response against a cell expressing a polypeptide of claim 1 in a subject, the method comprising
administering to the subject a therapeutically effective amount of the polypeptide of claim 1, or a polynucleotide encoding the polypeptide, thereby producing the immune response.

27. The method of claim 26, wherein the immune response is a T cell response.

28. The method of claim 26, wherein the immune response is a B cell response.

29. The method of claim 26, wherein the subject has breast cancer or prostate cancer.

30. The method of claim 29, wherein the immune response decreases the growth of the breast or prostate cancer.

31. A method for inhibiting the growth of a malignant cell expressing the polypeptide of claim 1, the method comprising,
(i) culturing cytotoxic T lymphocytes (CTLs) or CTL precursor cells with the polypeptide of claim 1 to produce activated CTLs or CTL precursors that recognize a 68h05-expressing cell, and
(ii) contacting the malignant cell with the activated CTLs or CTLs matured from the CTL precursors,
thereby inhibiting the growth of the malignant cell.

32. A method for inhibiting the growth of a malignant cell, comprising:
contacting the malignant cell with an effective amount of a cell-growth inhibiting molecule, wherein the cell growth inhibiting molecule comprises an antibody which specifically binds a polypeptide comprising an amino acid sequence set forth as SEQ ID NO: 1 or an immunogenic fragment thereof comprising at least eight consecutive amino acids 1-64 of SEQ ID NO: 1, amino acids 173-190 of SEQ ID NO: 1, amino acids 215-228 of SEQ ID NO: 1, 290-318 of SEQ ID NO: 1, or amino acids 261-334 of SEQ ID NO: 1, wherein the antibody is covalently linked to an effector molecule which inhibits the growth of cells,
thereby inhibiting the growth of the malignant cell.

33. The method of claim 32, wherein said antibody is a monoclonal antibody.

34. The method of claim 32, wherein the effector molecule is a chemotherapeutic agent.

35. The method of claim 32, wherein the effector molecule comprises a toxic moiety.

36. The method of claim 35, wherein the toxic moiety is selected from the group consisting of ricin A, abrin, diphtheria toxin or a subunit thereof, Pseudomonas exotoxin or a portion thereof, saporin, restrictocin or gelonin.

37. The method of claim 35, wherein the Pseudomonas exotoxin is selected from the group consisting of PE35, PE37, PE38, and PE40.

38. The method of claim 35, wherein the malignant cell is in vivo.

39. A pharmaceutical composition comprising a therapeutically effective amount of the polypeptide of claim 1 in a pharmaceutically acceptable carrier.

40. A pharmaceutical composition comprising a therapeutically effective amount of the polynucleotide of claim 6 in a pharmaceutically acceptable carrier.

41. A pharmaceutical composition comprising a therapeutically effective amount of the antibody of claim 12 in a pharmaceutically acceptable carrier.

42. A method for reducing the number of breast or prostate cancer cells in a subject, comprising
administering to the subject a therapeutically effective amount of the polypeptide of claim 1, wherein the administration of the polypeptide results in an immune response,
thereby reducing the number of breast or prostate cancer cells in the subject.

43. A method for reducing the number of breast or prostate cancer cells in a subject, comprising
administering to the subject a therapeutically effective amount of the polynucleotide of claim 6, wherein the administration of the polynucleotide results in an immune response,
thereby reducing the number of breast or prostate cancer cells in the subject.

44. A method for reducing the number of breast or prostate cancer cells in a subject, comprising
administering to the subject a therapeutically effective amount of the antibody of claim 16,
thereby reducing the number of breast or prostate cancer cells in the subject.

45. The method of claim 44, wherein the antibody is a humanized monoclonal antibody.

46. The method of claim 44, wherein the antibody is conjugated to a radionucleotide.

47. A kit for detecting an polynucleotide encoding 68h05 in a sample, comprising
an isolated nucleic acid sequence of at least ten nucleotides in length that specifically binds to the nucleic acid of claim 6; and
instructions for the use of the isolated nucleic acid sequence.

48. A kit for detecting a 68h05 polypeptide in a sample, comprising an monoclonal antibody that specifically binds to the polypeptide of claim 1; and
instructions for use of the antibody.


SEQUENCE LISTING (12 sequences)

SEQ ID NO: 1 - protein, 334 aa, Homo sapiens
MRGPSWSRPRPLLLLLLLLSPWPVWAQVSATASPSGSLGAPDCPEVCTCVPGGLASCSALSLPAVPPGLSLRLRALLLDHNRVRALPPGAFAGAGALQRLDLRENGLHSVHVRAFWGLGALQLLDLSANQLEALAPGTFAPLRALRNLSLAGNRLARLEPAALGALPLLRSLSLQDNELAALAPGLLGRLPALDALHLRGNPWGCGCALRPLCAWLRRHPLPASEAETVLCVWPGRLTLSPLTAFSDAAFSHCAQPLALRDLAVVYTLGPASFLVSLASCLALGSGLTACRARRRRLRTAALRPPRPPDPNPDPDPHGCASPADPGSPAAAAQA

SEQ ID NO: 2 - DNA, 1439 bp, Homo sapiens
ctttgcggagcctgggtggggtcgagacgggtgaggagcaggacctctgggttgcattgcgtgtgggcgcctgttgcagccgtgagctgggcgggtgcaggtgggcggtgtcctgctgctcaggtcagggctcggcgggcgcccggtggtggacaaggcggggtgaggtggtcttgatagcgctgtggccggcgctgagccaagagtagtttggagctcccggacaggtggctggcggtgccgggcgttggctgtttgccctgcccccgccccaagcgtggatccagccgtgggacaggaactgcctgagctcgggccagagagttaatatttgtgcaggcgccgcaggaacgggctccgcggacgacgggctccagggacgcacaggcagcgggcctcccaccgcgggtgccgggggcgggggggctgcccccatgcggggcccttcctggtcgcggcctcggccgctgctgctgctgttgctgctgctgtcgccttggcctgtctgggcccaggtgtcggccacggcctcgccctcggggtccctgggcgccccggactgccccgaggtgtgcacgtgcgtgccgggaggcctggccagctgctcggcactctcgctgcccgccgtgcccccgggcctgagcctgcgcctgcgcgcgctgctgctggaccacaaccgcgtccgtgcgctgccgccaggtgccttcgcgggagcgggcgcgctacagcgcctggacctgcgcgagaacgggctgcactcggtgcatgtgcgagccttctggggcctgggcgcgctgcagctgctggacctgagcgccaaccagctggaagcactggcaccagggactttcgcgccgctgcgcgcgctgcgcaacctctcattggccggcaaccggctggcgcgcctggagcccgcggcgctaggcgcgctcccgctgctgcgctcactcagcctgcaggacaacgagctggcggcactcgcgccggggctgctgggccgcctgcccgctctagacgcgctgcacctgcgcggcaacccttggggctgcgggtgcgcgctgcgcccgctctgcgcctggctgcgccggcacccgctgcccgcgtcagaggccgagacggtgctctgcgtgtggccgggacgcctgacgctcagccccctgactgccttttccgacgccgcctttagccattgcgcgcagccgctcgccctgcgggacctggccgtggtttacacgctcgggccggcctccttcctcgtcagcctggcttcctgcctggcgctgggctctgggctcaccgcctgccgtgcgcgccgccgccgcctccgcaccgccgccctccgcccgccgagaccgccagacccgaaccccgatcccgacccccacggctgtgcctcgcccgcggacccggggagccccgccgctgccgcccaagcctga

SEQ ID NO: 3 - protein, 9 aa, Homo sapiens
LLLLSPWPV

SEQ ID NO: 4 - protein, 9 aa, Homo sapiens
TLGPASFLV

SEQ ID NO: 5 - protein, 9 aa, Homo sapiens
ALLLDHNRV

SEQ ID NO: 6 - protein, 9 aa, Homo sapiens
GLLGRLPAL

SEQ ID NO: 7 - protein, 9 aa, Homo sapiens
FLVSLASCL

SEQ ID NO: 8 - DNA, 21 bp, Artificial Sequence
  Oligonucleotide primer.
ctctagacgcgctgcacctgc

SEQ ID NO: 9 - DNA, 19 bp, Artificial Sequence
  Oligonucleotide primer.
ccgctgccgcccaagcctg

SEQ ID NO: 10 - DNA, 17 bp, Artificial Sequence
  Oligonucleotide primer.
gcatgggtcagaaggat

SEQ ID NO: 11 - DNA, 17 bp, Artificial Sequence
  Oligonucleotide primer.
ccaatggtgatgacctg

SEQ ID NO: 12 - protein, 29 aa, Homo sapiens
CRARRRRLRTAALRPPRPPDPNPDPDPHG